SEQUENCE LISTING (30 sequences)

SEQ ID NO: 1 - DNA, 25 bp, B. burgdorferi
aattggtaccccaggatcccatatg

SEQ ID NO: 2 - DNA, 21 bp, B. burgdorferi
gcgggatccggtaccgtcgac

SEQ ID NO: 3 - protein, 203 aa, B. burgdorferi
MASWSHPQFEKGQREPGGHMARKESSKDSRSQLQVAGFKIGKESYGVSIEHIREIIKVPSEGVYAIPNVPEYIIGIYNLRGSIIPLINLNIKFGVPSISVTEEDMLLTGYLIVKIKNKLLGIFVDRVLKVISFDDSRVQEPPATLQTLDRKYISGVVKLDEADNLESEYLVLIDIAKIFDKCEFDDIPYKDQYEEVEHHHHHH

SEQ ID NO: 4 - protein, 191 aa, B. burgdorferi
MGSSHHHHHHSSGLVPRGSHTMDEKTTGWRGGHVVEGLAGELEQLRARLEHHPQGQREPGGHMANLPEEPNPPIIPTLKSLAKYETQLSEYVIYLVTFLAKTKVKVNDPNYPEYPYPDLSTLKDEHSITAVKHNINIYLEYIKKTKPIAEKVYNKYSQLKMVDGTGSEFELRRQACGRTRAPPPPPLRSGC

SEQ ID NO: 5 - protein, 216 aa, B. burgdorferi
MASWSHPQFEKGQREPGGHMAGKVVKVNAAGAAAKGGEETSVNGIASGIKGIVTAAEKAGEEGKLKSEAAGDGEANEDAGKLFAKKNDAGGDAKDAEKAAAAVSAVSGKQILKAIVDAAGKEEKGVVADVKDAKNPIEAAIGSTGEQNAAAFSHMKKNDQIAAAIVLRGMAKDGEFALKNDANNAEKGLKSTVESAVNKTVVDGQKKWVEHHHHHH

SEQ ID NO: 6 - protein, 286 aa, B. burgdorferi
MASWSHPQFEKGQREPGGHMSINKEQKTKEKTSEKQESEKQNIEKQEPEKQKQNAAKIIPTVSIQTVEIRESNQIPKSIEKYYKQAYPIQTFTLDFSITREKEFLKPEDKILPTQGKVESLSILINKKLLDFKAPENPKSSTLKNFKEIKNIENFFQNQDLLFVLTLKDKNNNNTINIMLNPPNDIQKPKDYILKDLKDTIKKGTGEKYLNPIYRFQIKNKKDYHSIDYNKVTISEKTIELDLLPHEQVFQMNKNFTKILDTITDLNNLKLVIQKELVVEHHHHHH

SEQ ID NO: 7 - protein, 892 aa, B. burgdorferi
MASWSHPQFEKGQREPGGHMAASDKGVKGAEQNLEKKVKGFLDTKKEELIGGLKTLGVEISPKVKELMQADEGAQGQAEEQVAQGPSEGSKLQEEIKQKIKELKEKIDKLLDEKAPIGKYCEHEEEIKKIRKQLEDIGDKLEDKGKLEIKLEELEDSLTKKKETRKKALEKVKEKLNEFKEQIGSASGQSDAEKVKEQGKIGLQAWQCVNSLGLGIAMDSKIDNNSGELANKVINDSLKKIEEELNKIGEEKKEVDGTGSHMADDQDKLLIDEEETVQIKDLNRVKIVNNNDLLLLDDGVASSNAITFKDFLETTKDKTFKGEGLSYFKEIIKSTIAEELAANTDFVETIYTKIINKLINNDSTNLSSLYRKIRSSLESGISSTTLSKDDYLLTLRLFGTIEKTPVPKQLLGLPSQYLPIGNKTSGTTFHPYEYKGKRTIINMDDYGDVTLYCNKSSDDEPIYLDIDIDIKHQRNGSKALYVGYSDESQKNLVYLHQGSSSVSIRVPMYKGWYIQKRTHISGNSVPYFCKLVDGTPGSHMGTIKDKSTEISKLLRTDKDKTKNQDRIELGEDNFVSKNNMSTTDTGITSLGSLNNLDLINRSQRVSEPPIISNEKAIATQAKVDLMNNINVTIINPKPAQNLGNSLNNTTTEDSVKFLSIENQEWLISKKILPSKLENLESFLKTQHEKEAFKTAKTIQSLISNSNMGKEIIKFKEEYYKLYNLFEGIQQKFHSQRNSFIKDTKFGENRQKNAVIFKSFSSIEKEIRDLNYKLSEIQSNFQIADVSWNNANSLLKESIEKLIQAIEKRYDNESRKQGQIGGPANRWDKNQADNFAKDAKYKAEHSANDLENAANYFRYSCSNEKEAKKLLEEIKKRFVRIGISLVEHHHHHH

SEQ ID NO: 8 - protein, 590 aa, B. burgdorferi
MGSSHHHHHHSSGLVPRGSHTMDEKTTGWRGGHVVEGLAGELEQLRARLEHHPQGQREPGGHMAAHELKEFANIIQDINKLASVASIFNSFSAIGGALDIVSDHLYFKKDNLDKLDIADLEILKNSFEQILYIKGSVAGKAKKLLLDYKNLKTDINKLKSYSNELVNGIKQQALEAENLEELIVSKYKLVDGTGSHMACNNSGKDGNTSANSADESVKGPNLTEISKKITESNAVVLAVKEIETLLASIDELATKAIGKKIQQNGGLAVEAGHNGTLLAGAYTISKLITQKLDGLKNSEKLKEKIENAKKCSEDFTKKLEGEHAQLGIENVTDENAKKAILITDAAKDKGAAELEKLFKAVENLAKAAKEMLANSVKELTSPIVAESPKKPVDGTPGSHMAGKVVKVNAAGAAAKGGEETSVNGIASGIKGIVTAAEKAGEEGKLKSEAAGDGEANEDAGKLFAKKNDAGGDAKDAEKAAAAVSAVSGKQILKAIVDAAGKEEKGVVADVKDAKNPIEAAIGSTGEQNAAAFSHMKKNDQIAAAIVLRGMAKDGEFALKNDANNAEKGLKSTVESAVNKTVVDGQKKWVE

SEQ ID NO: 9 - protein, 782 aa, B. burgdorferi
MGSSHHHHHHSSGLVPRGSHTMDEKTTGWRGGHVVEGLAGELEQLRARLEHHPQGQREPGGHMAAKDKITSSSSKVDELARKLQEEDKIKGVEENNKDELMQGDDPNSGVINSSPVLPENSQDNSPILKAAEQSGGQREEKVKKVEESEAKVEGKEEKQENTEERNKQELAKQEEEQQKRKAEKEKQKREEEQERKAKAEKEAKEKAERQKQEEQQKRKAEKEREEQRKEAEKRQVDNEIRTLTGKIDEINRNIDVIKEQTSVGAQGVIDRITGPVYDDFTDGNKAIYKTWGDLEDDNDEGLGKLLKELSDTRHNLRTKLNEGNKAYIIDTRSTEPQLKENVSVSEIKSDLDELKSKLEEVKEYLEDKDNFEEIKEYVAGSEDNYDEEDVDGTGSHMARENTKIVAICSIKGGVGKSTSAIIFSTLLSKKYKVLLIDADPQASTTSYFSDLLEEQGVDVSKQNIYEVLADKKNINSSTFRLNNNLYILPSYIYLYLFYDDNIPFKETRLKDSLKLLKHKYDYIIIDTSPSLGIILTNVLVVSNYIIIPMTAQKWSVESMQLLEFALKRLKLKIPIFPMVTNFKKNNTYKHLLELISRDDNFLGVIHEREDLNKRIAENDTFDLNKDYIKEYEITLEKFFQLSKKFLISVDGTPGSHMAFVVVDNGFLMNFKNVIFNGIDDAKLYDQRDMVYGGFRYSKEAYFQIIGNYDVKLNKMKQYTPAIVVNVFKININDALFNSLLKQKTLKVTLISHNNKEYILQTNNFLSKYNFQTPEKENSSYVE

SEQ ID NO: 10 - protein, 745 aa, B. burgdorferi
MASWSHPQFEKGQREPGGHMARENTKIVAICSIKGGVGKSTSAIIFSTLLSKKYKVLLIDADPQASTTSYFSDLLEEQGVDVSKQNIYEVLADKKNINSSTFRLNNNLYILPSYIYLYLFYDDNIPFKETRLKDSLKLLKHKYDYIIIDTSPSLGIILTNVLVVSNYIIIPMTAQKWSVESMQLLEFALKRLKLKIPIFPMVTNFKKNNTYKHLLELISRDDNFLGVIHEREDLNKRIAENDTFDLNKDYIKEYEITLEKFFQLSKKFLISVDGTGSHMAFVVVDNGFLMNFKNVIFNGIDDAKLYDQRDMVYGGFRYSKEAYFQIIGNYDVKLNKMKQYTPAIVVNVFKININDALFNSLLKQKTLKVTLISHNNKEYILQTNNFLSKYNFQTPEKENSSYVDGTPGSHMAAKDKITSSSSKVDELARKLQEEDKIKGVEENNKDELMQGDDPNSGVINSSPVLPENSQDNSPILKAAEQSGGQREEKVKKVEESEAKVEGKEEKQENTEERNKQELAKQEEEQQKRKAEKEKQKREEEQERKAKAEKEAKEKAERQKQEEQQKRKAEKEREEQRKEAEKRQVDNEIRTLTGKIDEINRNIDVIKEQTSVGAQGVIDRITGPVYDDFTDGNKAIYKTWGDLEDDNDEGLGKLLKELSDTRHNLRTKLNEGNKAYIIDTRSTEPQLKENVSVSEIKSDLDELKSKLEEVKEYLEDKDNFEEIKEYVAGSEDNYDEEDVEHHHHHH

SEQ ID NO: 11 - protein, 782 aa, B. burgdorferi
MGSSHHHHHHSSGLVPRGSHTMDEKTTGWRGGHVVEGLAGELEQLRARLEHHPQGQREPGGHMARENTKIVAICSIKGGVGKSTSAIIFSTLLSKKYKVLLIDADPQASTTSYFSDLLEEQGVDVSKQNIYEVLADKKNINSSTFRLNNNLYILPSYIYLYLFYDDNIPFKETRLKDSLKLLKHKYDYIIIDTSPSLGIILTNVLVVSNYIIIPMTAQKWSVESMQLLEFALKRLKLKIPIFPMVTNFKKNNTYKHLLELISRDDNFLGVIHEREDLNKRIAENDTFDLNKDYIKEYEITLEKFFQLSKKFLISVDGTGSHMAAKDKITSSSSKVDELARKLQEEDKIKGVEENNKDELMQGDDPNSGVINSSPVLPENSQDNSPILKAAEQSGGQREEKVKKVEESEAKVEGKEEKQENTEERNKQELAKQEEEQQKRKAEKEKQKREEEQERKAKAGKEAKEKAERQKQEEQQKRKAEKEREEQRKEAEKRQVDNEIRTLTGKIDEINRNIDVIKEQTSVGAQGVIDRITGPVYDDFTDGNKAIYKTWGDLEDDNDEGLGKLLKELSDTRHNLRTKLNEGNKAYIIDTRSTEPQLKENVSVSEIKSDLDELKSKLEEVKEYLEDKDNFEEIKEYVAGSEDNYDEEDVDGTPGSHMAFVVVDNGFLMNFKNVIFNGIDDAKLYDQRDMVYGGFRYSKEAYFQIIGNYDVKLNKMKQYTPAIVVNVFKININDALFNSLLKQKTLKVTLISHNNKEYILQTNNFLSKYNFQTPEKENSSYVE

SEQ ID NO: 12 - protein, 633 aa, B. burgdorferi
MGSSHHHHHHSSGLVPRGSHTMDEKTTGWRGGHVVEGLAGELEQLRARLEHHPQGQREPGGHMARKESSKDSRSQLQVAGFKIGKESYGVSIEHIREIIKVPSEGVYAIPNVPEYIIGIYNLRGSIIPLINLNIKFGVPSISVTEEDMLLTGYLIVKIKNKLLGIFVDRVLKVISFDDSRVQEPPATLQTLDRKYISGVVKLDEADNLESEYLVLIDIAKIFDKCEFDDIPYKDQYEEVDGTGSHMANLPEEPKPPIIPTLKSLAKYETQLSEYVMYLVTFLAKTKVKVNDPNYPEYPYPDLSTLKDEHSITAVKHNINIYLEYIKKTKPIAEKVYNKYSQLKMVDGTPGSHMAKSNNTEAISELQSSPIKLGKIKVLQKTEKIVSTQNLQNLQQSQFFKNEKEKIIKKIAQEFDENEKLINKIGPNIEMFAQTINTDIQKIEPNDQFGINKTLFTEKKDNNIDFMLKDNRLRRLFYSSLNYDENKIKKLATILAQTSSSNDYHYTLIGLIFWTGFKIQEAFESAVNILTKDEQKRLIFNFRTKTVKEIQENFEKLMQERNSWIKIVDNIIGEYDKNTGGCKADGKILGEVIRVGYEHELDSNKSMQILNNIETPLKTCCDHIHYVEHHHHHH

SEQ ID NO: 13 - protein, 627 aa, B. burgdorferi
MGSSHHHHHHSSGLVPRGSHTMDEKTTGWRGGHVVEGLAGELEQLRARLEHHPQGQREPGGHMARKESSKDSRSQLQVAGFKIGKESYGVSIEHIREIIKVPSEGVYAIPNVPEYIIGIYNLRGSIIPLINLNIKFGVPSISVTEEDMLLTGYLIVKIKNKLLGIFVDRVLKVISFDDSRVQEPPATLQTLDRKYISGVVKLDEADNLESEYLVLIDIAKIFDKCEFDDIPYKDQYEEVDGTGSHMAKSNNTEAISELQSSPIKLGKIKVLQKTEKIVSTQNLQNLQQSQFFKNEKEKIIKKIAQEFDENEKLINKIGPNIEMFAQTINTDIQKIEPNDQFGINKTLFTEKKDNNIDFMLKDNRLRRLFYSSLNYDENKIKKLATILAQTSSSNDYHYTLIGLIFWTGFKIQEAFESAVNILTKDEQKRLIFNFRTKTVKEIQENFEKLMQERNSWIKIVDNIIGEYDKNTGGCKADGKILGEVIRVGYEHELDSNKSMQILNNIETPLKTCCDHIHYVDGTPGSHMANLPEEPNPPIIPTLKSLAKYETQLSEYVIYLVTFLAKTKVKVNDPNYPEYPYPDLSTLKDEHSITAVKHNINIYLEYIKKTKPIAEKVYNKYSQLKMVE

SEQ ID NO: 14 - protein, 626 aa, B. burgdorferi
MASWSHPQFEKGQREPGGHMSINDNNYKIFGQEVPIAKIIAFESTEEFEKKYEIKSLKLNSEESNIDFEQNRTGFAKINLKETSREPQYIYSYNFGVFDNSLADYFKLFYKKSKCNYMPAYLTIKDKQTNKDKTYEIILNLKLFNDAIRLIFDKYSNLSKEKLKLFIDEVDGTGSHMALNSKLSGNKEEQKNNNDIKEALNGVQENAINNLYGNKKEKKDFIKNSEKLKDKGLDVTTLPLEPVVAPSVESAVSLGESNNRIGIPTISIEHNQKKEIKEEDFFPSTEEEKQADKAIKDIENLIGESGFPELIENVCSLKHEYTLIRSDFYDVITKIQNKKISLMKNSHNNRNKIRELVQLQNNLKIGDELDKIMGCIDTAEQEIRSAAFFFDEAKESLKEGIIKRLEKSKNRAASQLSKKALNRAEDALRCLENYSSKKGEAIGRRSFIKEVVEQAKNALSKSVDGTPGSHMAKSFYNEHYHFTSFIYKGGFIMTQYFNDDIPCNSCNRLIRKYDQTCVACGAKNKQNKKSYYGLIAFLFCLFFGYLGFSNLYLGKNPKIGFTFLFISIVFLLLAVLLHKSNKTNYLLVLFVLLVYFFFVNRFKIYKFFKKVTRKIISLVEHHHHHH

SEQ ID NO: 15 - protein, 333 aa, B. burgdorferi
MGSSHHHHHHSSGLVPRGSHTMDEKTTGWRGGHVVEGLAGELEQLRARLEHHPQGQREPGGHMADDQEKLLIDEEETVQIKDLNKVTTVNDTDLLLLDDGAASSNAITFKNFLDASKDKIFKGEGLDYFKQIIKSTIAEELAADKDFVEKIYAKITDKLINNDSTNISNLFSKIKSRLTDSISSATLSRSDHLLIMPSSDTIQKTPVPKHILGVPSNFTYGSITRSTTLYPSDYENKAISINMEDNDDVTLIFYKNYDNDPIYLDIEIQVKINDNRMQKKSLKLMYSDEITYNWVYEITGPRGLFTRTPIYNGWYIQKRASLYGDSVPDLLKL

SEQ ID NO: 16 - protein, 212 aa, B. burgdorferi
MGSSHHHHHHSSGLVPRGSHTMDEKTTGWRGGHVVEGLAGELEQLRARLEHHPQGQREPGGHMAEENYTETKRAFSKEDFNLINKRLDNYDFKNEYEKSHVFSDAPRIRGDLRKIGIKEKSVFLDALEAIEYLIKIKISTDSIFLSEDMIRLIGSYPDSIFNYLIQLNSDKIDYAEKYGDNARNNFKKDYSEDKANTVKQILKQILADLPKD

SEQ ID NO: 17 - protein, 100 aa, B. burgdorferi
MGFKADPKKSEVKDYFDSLAKKLEETKKGLNDLNNNNNAGGGAADAGKAAEKGTAGKADGVQGAIAEISGWLEEMVKAATKVDGTGSEFELRRQACGRTR

SEQ ID NO: 18 - protein, 308 aa, B. burgdorferi
MASWSHPQFEKGQREPGGHMAQKGAESIGSQKENDLNLEDSSKKSHQNAKQDLPAVTEDSVSLFNGNKIFVSKEKNSSGKYDLRATIDQVELKGTSDKNNGSGTLEGSKPDKSKVKLTVSADLNTVTLEAFDASNQKISSKVTKKQGSITEETLKANKLDSKKLTRSNGTTLEYSQITDADNATKAVETLKNSIKLEGSLVGGKTTVEIKEGTVTLKREIEKNGKVKVFLNDTAGSNKKTGKWEDSTSTLTISADSKKTKDLVFLTDGTITVQQYNTAGTSLEGSASEIKNLSELKNALKVEHHHHHH

SEQ ID NO: 19 - protein, 664 aa, B. burgdorferi
MASWSHPQFEKGQREPGGHMANLNEDYKNKVKGILNKAADDQETTSADTNSNAAKNIPIADNDKVAAELKKQSQAAKTVAAAPNKGSQNQPQTTPNKGSQNQQAAPSPQLQSLSFSADLSNLPKTTAARAASLTKQRIPIQAVTTVPGNTRTFNSRNSGLPTFALNYSFSQPTRQQTNSSSAVQTTTSSGSKLQTLKNELIRAISEEKNKTQNNFGFRETYDQFKMKDSAFELLDVISSAKVYDRSYAPQLNSNTPEAENERNKFYALMDFDQYKIEQFGSIMEALYNENQNHSLIRELMISGLGTQISFELALEEINKKIEIFNQDYLNAKINSFDFTMKLKELKSKLNQILDKRKEWSRQADGLIANASSNSSLSDSKSLAEYIKKRYLDNMQNARQSVLEAYISIMVDGTGSHMAEENYTETKRAFSKEDFNLINKRLDNYDFKNEYEKSHVFSDAPRIRGDLRKIGIKEKSVFLDALEAIEYLIKIKISTDSIFLSEDMIRLIGSYPDSIFNYLIQLNSDKIDYAEKYGDNARNNFKKDYSEDKANTVKQILKQILADLPKDVDGTPGSHMAKKIALIAHDKKKEDLVNFVKQNYLFLSKFKLIATGTTGSKIQQATDLTIFKYKSGPMGGDQQIGAEVAEGNILAIFFFRDVEHHHHHH

SEQ ID NO: 20 - protein, 612 aa, B. burgdorferi
MASWSHPQFEKGQREPGGHMGLKKETLDTLTVSDLKKLKNSFEKLLSIKTDISKMLEQLLLDYKDYINSTKTDIAKLESHLTELYKQIVKKSFQTTELKNNILSISNLVDGTGSHMAVNSYTNTKEDTENLKNASGDINSLSQKSLKEVTIAKLKEFGDKLTAQKNEETTKIEKITAEKESDLLETLDSKIQPVYFTKPETTEETKNNVKMQIKRIIYSSLDYDKGEIKKLKEIIEKVKGKDGGEYMIIDLLYGKALDIQTQIDKHLEPTKEDNLNTLSEENLKELLIHVEFDLILKEKFKKALKKTVGEVPQEIQKIEKETNIKVDGTPGSHMADDQEKLLIDEEETVQIKDLNKVTTVNDTDLLLLDDGAASSNAITFKNFLDASKDKIFKGEGLDYFKQIIKSTIAEELAADKDFVEKIYAKITDKLINNDSTNISNLFSKIKSRLTDSISSATLSRSDHLLIMPSSDTIQKTPVPKHILGVPSNFTYGSITRSTTLYPSDYENKAISINMEDNDDVTLIFYKNYDNDPIYLDIEIQVKINDNRMQKKSLKLMYSDEITYNWVYEITGPRGLFTRTPIYNGWYIQKRASLYGDSVPDLLKLLEHHHHHH

SEQ ID NO: 21 - protein, 652 aa, B. burgdorferi
MGSSHHHHHHSSGLVPRGSHTMDEKTTGWRGGHVVEGLAGELEQLRARLEHHPQGQREPGGHMGSSGAFLDGICRLPLLKNELNFIHGTGHGVGFFLNVHELPVSISPNSSYLFKGSEVVSIEPGLYRTFSHGIRIENLVFVRQAFTNDFGAFLEFENLTLVPFEKELIVKEMLSEDELNYINDYHECVFLTLKERFDNEGELEFLAKLTSKIVDGTGSHMAQKGAESIGSQKENDLNLEDSSKKSHQNAKQDLPAVTEDSVSLFNGNKIFVSKEKNSSGKYDLRATIDQVELKGTSDKNNGSGTLEGSKPDKSKVKLTVSADLNTVTLEAFDASNQKISSKVTKKQGSITEETLKANKLDSKKLTRSNGTTLEYSQITDADNATKAVETLKNSIKLEGSLVGGKTTVEIKEGTVTLKREIEKNGKVKVFLNDTAGSNKKTGKWEDSTSTLTISADSKKTKDLVFLTDGTITVQQYNTAGTSLEGSASEIKNLSELKNALKVDGTPGSHMAAEEITSTLKVPNGFKVEIFLNNTIEKPRGITSDQDGNIFIGSGSTFAYFVTKNRKIYTIAKTLQKPIGIDYWDNKLYISSVDKIYVVKNVKEEINKSIKSHKDYTWKMQIFAFLPKNNSQMHSGRYIKVDSKNNKLIVNVE

SEQ ID NO: 22 - protein, 652 aa, B. burgdorferi
MGSSHHHHHHSSGLVPRGSHTMDEKTTGWRGGHVVEGLAGELEQLRARLEHHPQGQREPGGHMAQKGAESIGSQKENDLNLEDSSKKSHQNAKQDLPAVTEDSVSLFNGNKIFVSKEKNSSGKYDLRATIDQVELKGTSDKNNGSGTLEGSKPDKSKVKLTVSADLNTVTLEAFDASNQKISSKVTKKQGSITEETLKANKLDSKKLTRSNGTTLEYSQITDADNATKAVETLKNSIKLEGSLVGGKTTVEIKEGTVTLKREIEKNGKVKVFLNDTAGSNKKTGKWEDSTSTLTISADSKKTKDLVFLTDGTITVQQYNTAGTSLEGSASEIKNLSELKNALKVDGTGSHMAAEEITSTLKVPNGFKVEIFLNNTIEKPRGITSDQDGNIFIGSGSTFAYFVTKNRKIYTIAKTLQKPIGIDYWDNKLYISSVDKIYVVKNVKEEINKSIKSHKDYTWKMQIFAFLPKNNSQMHSGRYIKVDSKNNKLIVNVDGTPGSHMGSSGAFLDGICRLPLLKNELNFIHGTGHGVGFFLNVHELPVSISPNSSYLFKGSEVVSIEPGLYRTFSHGIRIENLVFVRQAFTNDFGAFLEFENLTLVPFEKELIVKEMLSEDELNYINDYHECVFLTLKERFDNEGELEFLAKLTSKIVE

SEQ ID NO: 23 - protein, 711 aa, B. burgdorferi
MGSSHHHHHHSSGLVPRGSHTMDEKTTGWRGGHVVEGLAGELEQLRARLEHHPQGQREPGGHMATVEADVILEDGTYGRAAVPSGASTGINEAVELRDGDKSVYMGKGVLKAIENIKNIIAPELEGMSALNQVAIDRKMLELDGTPTKEKLGANAILAVSMATAKAAAKYLGLRPYQYLGAYKANILPTPMCNIINGGAHSDNSVDFQEFMIMPIGAKTFSEAIRMAAEVFHTLKGILSGKGYATSVGDEGGFAPNLKSNEEACEVIIEAIKKAGYEPGKDIAIALDPATSELYDPKTKKYVLKWSTKEKLTSEQMVEYWAKWVEKYPIISIEDGMAEEDWDGWKKLTDKIGNKIQLVGDDLFVTNTSFLKKGIEMGVANSILIKVNQIGTLTETFEAVEMAKKAGYTAIVSHRSGETEDTTIADLVVALGTGQIKTGSLSRTDRIAKYNQLIRIEEELETTAEYHGKSVFYSIKQKVDGTGSHMGFKADPKKSEVKDYFDSLAKKLEETKKGLNDLNNNNNAGGGAADAGKAAEKGTAGKADGVQGAIAEISGWLEEMVKAATKVDGTPGSHMAGKVVKVNAAGAAAKGGEETSVNGIASGIKGIVTAAEKAGEEGKLKSEAAGDGEANEDAGKLFAKKNDAGGDAKDAEKAAAAVSAVSGKQILKAIVDAAGKEEKGVVADVKDAKNPIEAAIGSTGEQNAAAAFSHVE

SEQ ID NO: 24 - protein, 674 aa, B. burgdorferi
MASWSHPQFEKGQREPGGHMAGKVVKVNAAGAAAKGGEETSVNGIASGIKGIVTAAEKAGEEGKLKSEAAGDGEANEDAGKLFAKKNDAGGDAKDAEKAAAAVSAVSGKQILKAIVDAAGKEEKGVVADVKDAKNPIEAAIGSTGEQNAAAAFSHVDGTGSHMATVEADVILEDGTYGRAAVPSGASTGINEAVELRDGDKSVYMGKGVLKAIENIKNIIAPELEGMSALNQVAIDRKMLELDGTPTKEKLGANAILAVSMATAKAAAKYLGLRPYQYLGAYKANILPTPMCNIINGGAHSDNSVDFQEFMIMPIGAKTFSEAIRMAAEVFHTLKGILSGKGYATSVGDEGGFAPNLKSNEEACEVIIEAIKKAGYEPGKDIAIALDPATSELYDPKTKKYVLKWSTKEKLTSEQMVEYWAKWVEKYPIISIEDGMAEEDWDGWKKLTDKIGNKIQLVGDDLFVTNTSFLKKGIEMGVANSILIKVNQIGTLTETFEAVEMAKKAGYTAIVSHRSGETEDTTIADLVVALGTGQIKTGSLSRTDRIAKYNQLIRIEEELETTAEYHGKSVFYSIKQKVDGTPGSHMGFKADPKKSEVKDYFDSLAKKLEETKKGLNDLNNNNNAGGGAADAGKAAEKGTAGKADGVQGAIAEISGWLEEMVKAATKVEHHHHHH

SEQ ID NO: 25 - protein, 602 aa, B. burgdorferi
MASWSHPQFEKGQREPGGHMAQKGAESIGSQKENDLNLEDSSKKSHQNAKQDLPAVTEDSVSLFNGNKIFVSKEKNSSGKYDLRATIDQVELKGTSDKNNGSGTLEGSKPDKSKVKLTVSADLNTVTLEAFDASNQKISSKVTKKQGSITEETLKANKLDSKKLTRSNGTTLEYSQITDADNATKAVETLKNSIKLEGSLVVGKTTVEIKEGTVTLKREIEKDGKVKVFLNDTAGSNKKTGKWEDSTSTLTISADSKKTKDLVFLTDGTITVQQYNTAGTSLEGSASEIKNLSELKNALKGHPMGNNSGKGGNTSANSADESVKGPNLTEISKKITDSNAVLLAVKEVEALLSSIDEIAAKAIGKKIHQNNGLDTEYNHNGSLLAGAYAISTLIKQKLDGLKNEGLKEKIDAAKKCSETFTNKLKEKHTDLGKEGVTDADAKEAILKTNGTKTKGAEELGKLFESVEVLSKAAKEMLANSVKELTSPVVAESPKKPGTMASQNVRTAEELGMQPAKINTPASLSGSQASWTLRVHVGANQDEAIAVNIYAANVANLFSGEGAQTAQAAPVQEGVQQEGAQQPAPATAPSQGGVGHHHHHHNM

SEQ ID NO: 26 - protein, 799 aa, B. burgdorferi
MASWSHPQFEKGQREPGGHMAQKGAESIGSQKENDLNLEDSSKKSHQNAKQDLPAVTEDSVSLFNGNKIFVSKEKNSSGKYDLRATIDQVELKGTSDKNNGSGTLEGSKPDKSKVKLTVSADLNTVTLEAFDASNQKISSKVTKKQGSITEETLKANKLDSKKLTRSNGTTLEYSQITDADNATKAVETLKNSIKLEGSLVVGKTTVEIKEGTVTLKREIEKDGKVKVFLNDTAGSNKKTGKWEDSTSTLTISADSKKTKDLVFLTDGTITVQQYNTAGTSLEGSASEIKNLSELKNALKGHPMGNNSGKGGNTSANSADESVKGPNLTEISKKITDSNAVLLAVKEVEALLSSIDEIAAKAIGKKIHQNNGLDTEYNHNGSLLAGAYAISTLIKQKLDGLKNEGLKEKIDAAKKCSETFTNKLKEKHTDLGKEGVTDADAKEAILKTNGTKTKGAEELGKLFESVEVLSKAAKEMLANSVKELTSPVVAESPKKPGTPGSHMAGKVVKVNAAGAAAKGGEETSVNGIASGIKGIVTAAEKAGEEGKLKSEAAGDGEANEDAGKLFAKKNDAGGDAKDAEKAAAAVSAVSGKQILKAIVDAAGKEEKGVVADVKDAKNPIEAAIGSTGEQNAAAFSHMKKNDQIAAAIVLRGMAKDGEFALKNDANNAEKGLKSTVESAVNKTVVDGQKKWVDGTMASQNVRTAEELGMQPAKINTPASLSGSQASWTLRVHVGANQDEAIAVNIYAANVANLFSGEGAQTAQAAPVQEGVQQEGAQQPAPATAPSQGGVGHHHHHHNM

SEQ ID NO: 27 - protein, 425 aa, B. burgdorferi
MGSSHHHHHHSSGLVPRGSHTMASWSHPQFEKGGHMACNNSGKDGNTSANSADESVKGPNLTEISKKITDSNAVLLAVKEVEALLSSIDELAKAIGKKIKNDGSLDNEANRNESLLAGAYTISTLITQKLSKLNGSEGLKEKIAAAKKCSEEFSTKLKDNHAQLGIQGVTDENAKKAILKANAAGKDKGVEELERLSGSLESLSKAAKEMLANSVKELTSPVVVESPKKPAMACNNSGKDGNASANSADESVKGPNLTEISKKITESNAVVLAVKEVETLLASINQLAKAIGKKIDQNGTLGDDGGQNGSLLAGAYAISTVIIEKLSTLKNVEELKEKITKAKDCSEKFAGKLKNEHASLGKKDATDDDAKKAILKTHGNTDKGAKELKDLSDSVESLVKAAKEMLTNSVKELTSPVVAESPKKP

SEQ ID NO: 28 - protein, 425 aa, B. burgdorferi
MGSSHHHHHHSSGLVPRGSHTMASWSHPQFEKGGHMACNNSGKDGNTSANSADESVKGPNLTEISKKITDSNAVLLAVKEVEALLSSIDELAKAIGKKIKNDGSLDNEANRNESLLAGAYTISTLITQKLSKLNASEGLKAKIAAAAKCSEEFSTKLKDNHAQLGIQGVTDENAKKAILKANAAGKDKGVEELERLSGSLESLSKAAKEMLANSVKELTSPVVVESPKKPAMACNNSGKDGNASANSADESVKGPNLTEISKKITESNAVVLAVKEVETLLASINQLAKAIGKKIDQNGTLGDDGGQNGSLLAGAYAISTVIIEKLSTLKNVEELKEKITKAKDCSEKFAGKLKNEHASLGKKDATDDDAKKAILKTHGNTDKGAKELKDLSDSVESLVKAAKEMLTNSVKELTSPVVAESPKKP

SEQ ID NO: 29 - protein, 425 aa, B. burgdorferi
MGSSHHHHHHSSGLVPRGSHTMASWSHPQFEKGGHMACNNSGKDGNTSANSADESVKGPNLTEISKKITDSNAVLLAVKEVEALLSSIDELAKAIGKKIKNDGSLDNEANRNESLLAGAYTISTLITQKLSKLNGSEGLKEKIAAAKKCSEEFSTKLKDNHAQLGIQGVTDENAKKAILKANAAGKDKGVEELERLSGSLESLSKAAKEMLANSVKELTSPVVVESPKKPAMACNNSGKDGNASANSADESVKGPNLTEISKKITESNAVVLAVKEVETLLASINQLAKAIGKKIDQNGTLGDSGGQNGALLAGAYAISTVIIEKLSTLKNVEELKEKITKAKDCSEKFAGKLKNEHASLGKKDATDDDAKKAILKTHGNTDKGAKELKDLSDSVESLVKAAKEMLTNSVKELTSPVVAESPKKP

SEQ ID NO: 30 - protein, 427 aa, B. burgdorferi
MGSSHHHHHHSSGLVPRGSHTMASWSHPQFEKGGHMACNNSGKDGNTSANSADESVKGPNLTEISKKITDSNAVLLAVKEVEALLSSIDELAKAIGKKIKNDGSLDNEANRNESLLAGAYTISTLITQKLSKLNASEGLKAKIAAAAKCSEEFSTKLKDNHAQLGIQGVTDENAKKAILKANAAGKDKGVEELERLSGSLESLSKAAKEMLANSVKELTSPVVVESPKKPAMACNNSGKDGNASANSADESVKGPNLTEISKKITESNAVVLAVKEVETLLASINQLAKAIGKKIDQNGTLGDSGGQNGALLAGAAAISTVIIEKLSTLKNVEELKEKITKAKDCSEKFAGKLKNEHASLGKKDATDDDAKKAILKTHGNTDKGAKELKDLSDSVESLVKAAKEMLTNSVKELTSPVVAESPKKPVV

(19) United States
(12) Patent Application Publication
(10) Pub. No.: US 2017/0212114 A1
(43) Pub. Date: Jul. 27, 2017

(54) Recombinant Borrelia Proteins And Methods Of Use Thereof

(71) Applicant: The Research Foundation for The State University of New York, Albany, NY (US)

(72) Inventors: Benjamin J. Luft, Riverhead, NY (US); John F. Bruno, Islip, NY (US); Yun Xu, Stony Brook, NY (US)

(21) Appl. No.: 15/481,438

(22) Filed: Apr. 6, 2017

Related U.S. Application Data
(63) Continuation-in-part of application No. PCT/US2015/054303, filed on Oct. 6, 2015.
(60) Provisional application No. 62/365,937, filed on Jul. 22, 2016.
(60) Provisional application No. 62/076,059, filed on Nov. 6, 2014.
(60) Provisional application No. 62/060,867, filed on Oct. 7, 2014.

(51) Int. Cl. G01N 33/569; C07K 14/20

(52) U.S. Cl. CPC G01N 33/56911; C07K 14/20; C07K 2319/70; G01N 2333/20; G01N 2800/26; G01N 2800/60

(57) ABSTRACT

Novel Borrelia burgdorferi recombinant proteins and methods of assessing a sample for the presence of antibodies to certain proteins of Borrelia burgdorferi, are described, as are methods of diagnosing Lyme disease. Microarrays of proteins of Borrelia burgdorferi are also described.

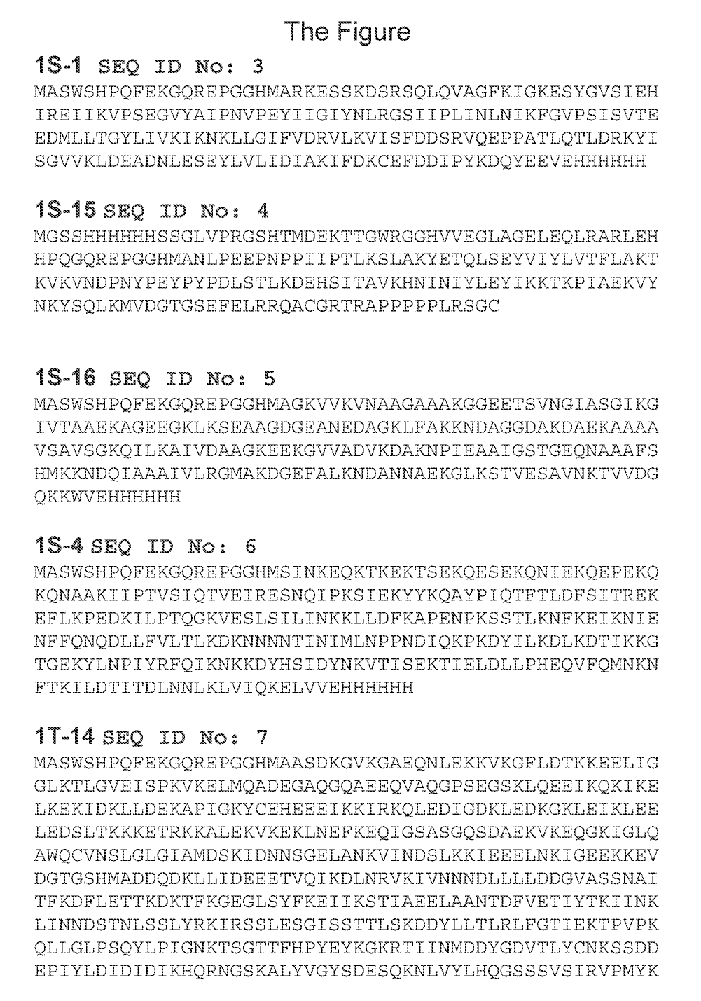

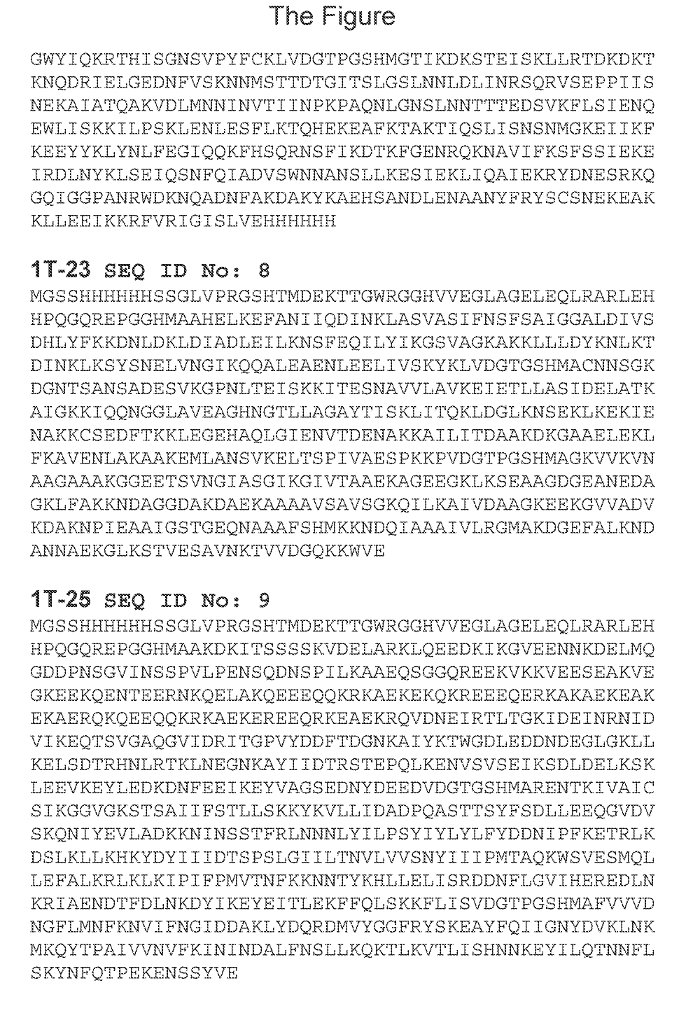

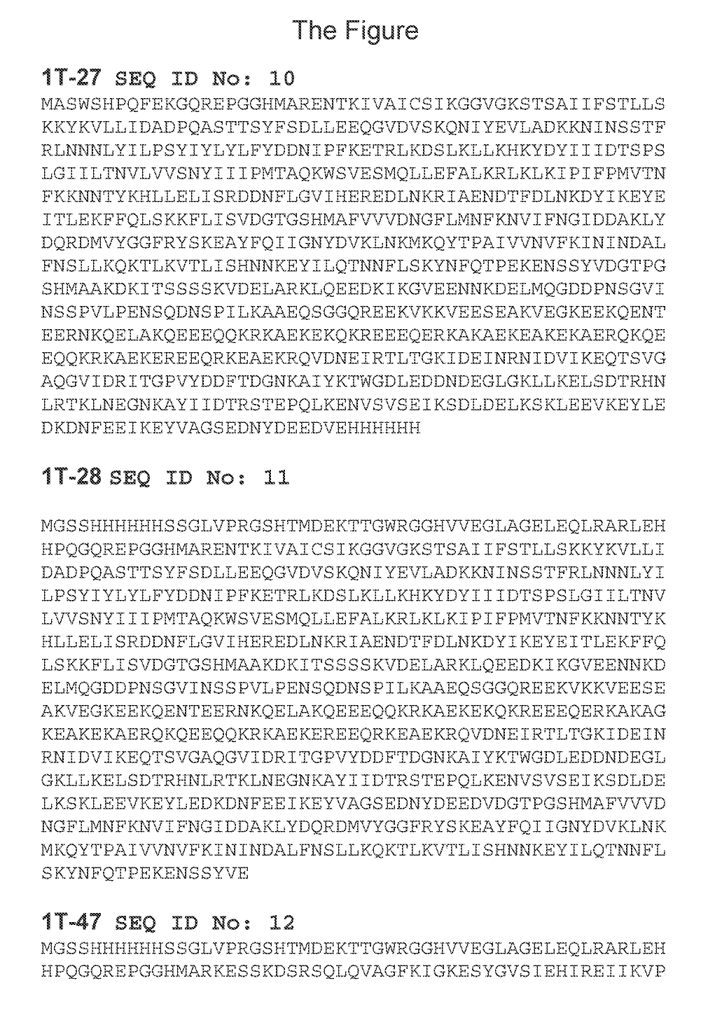

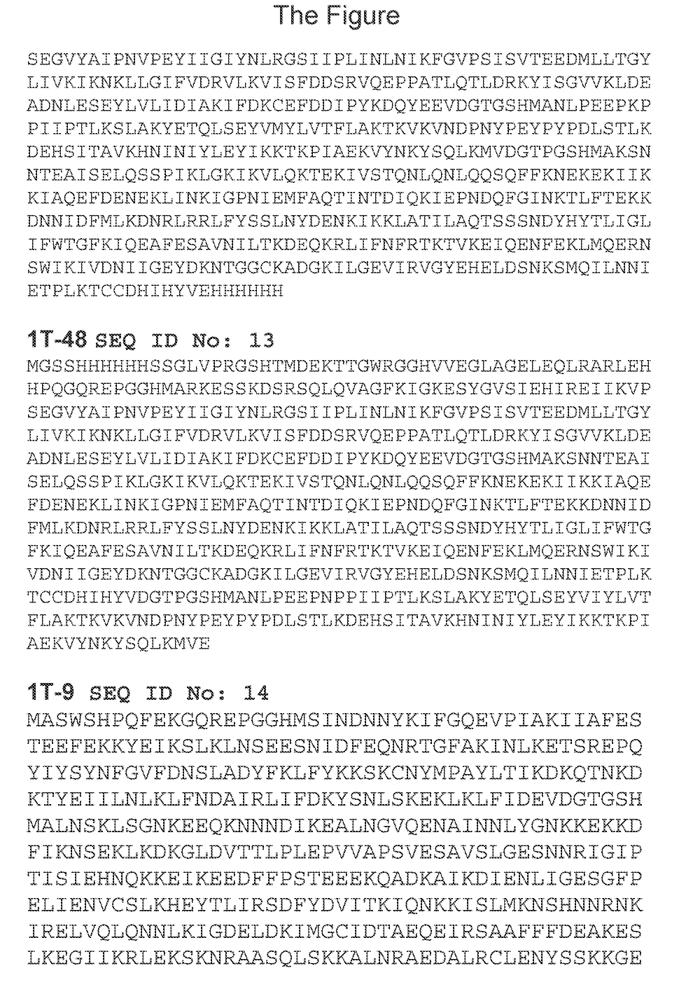

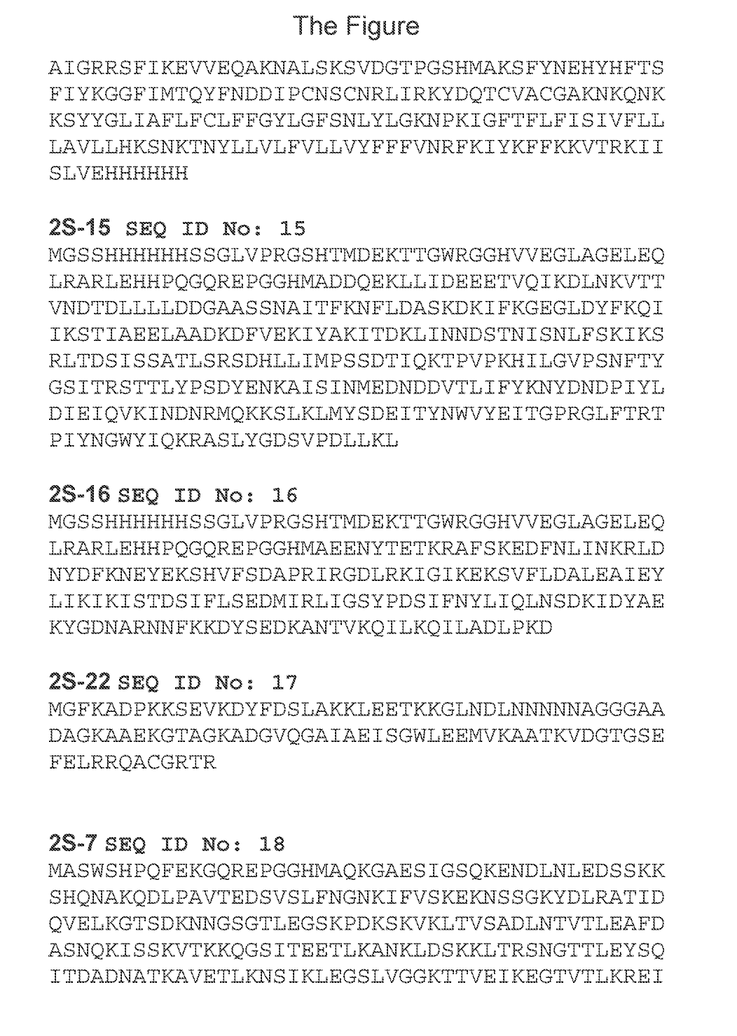

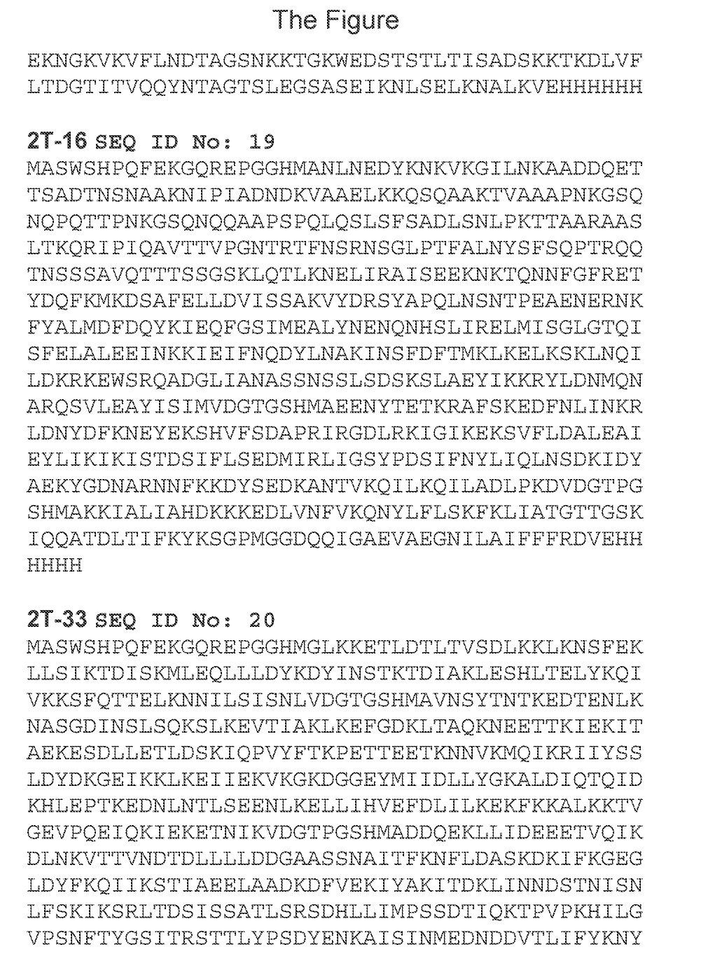

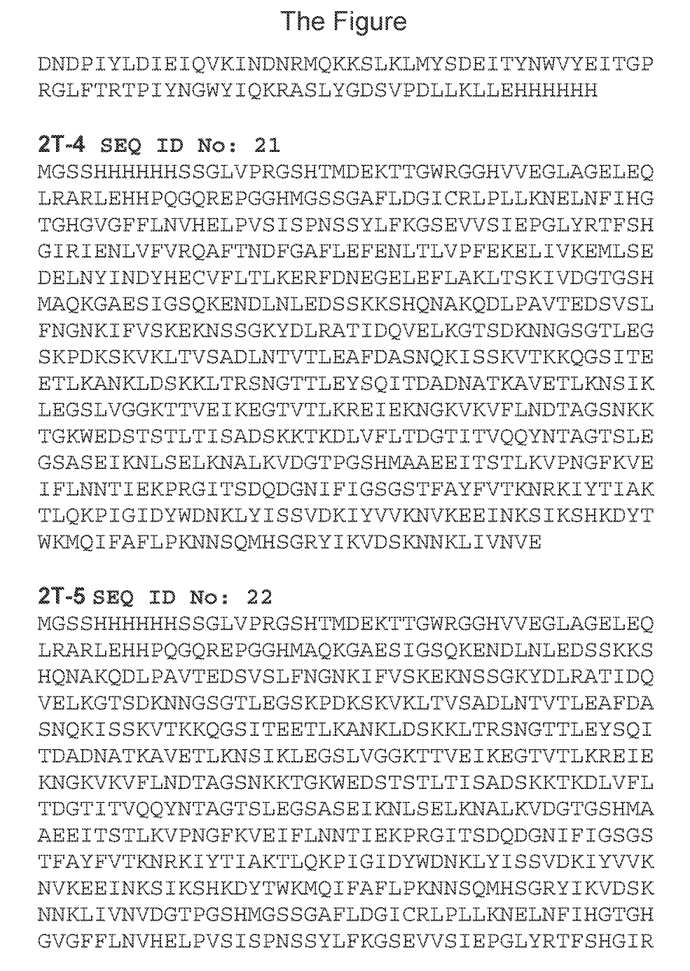

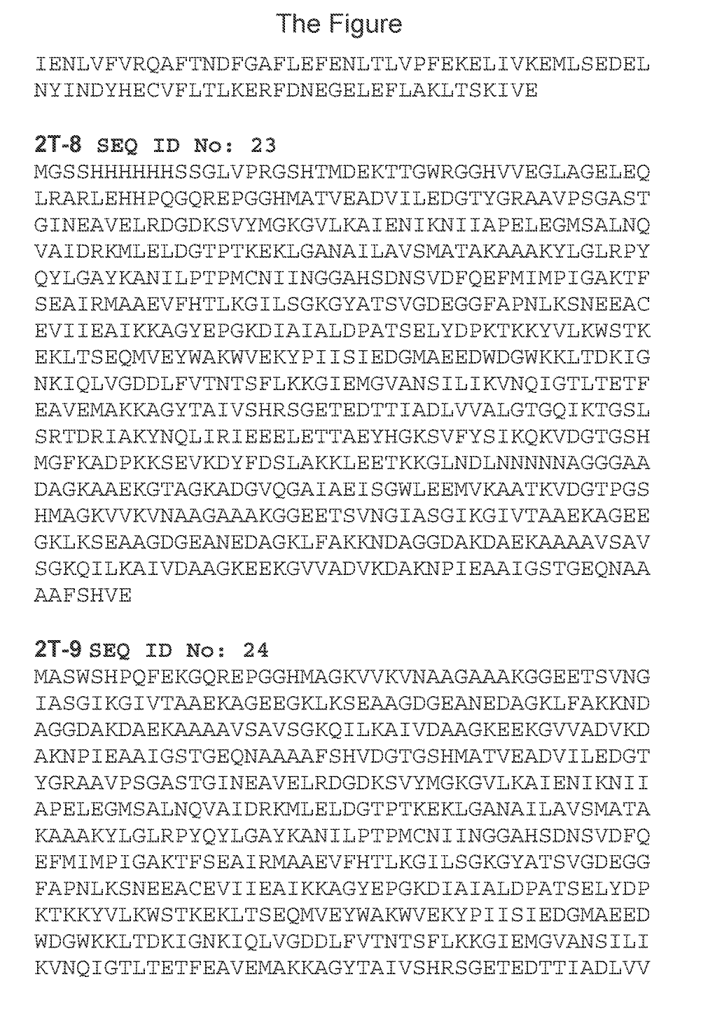

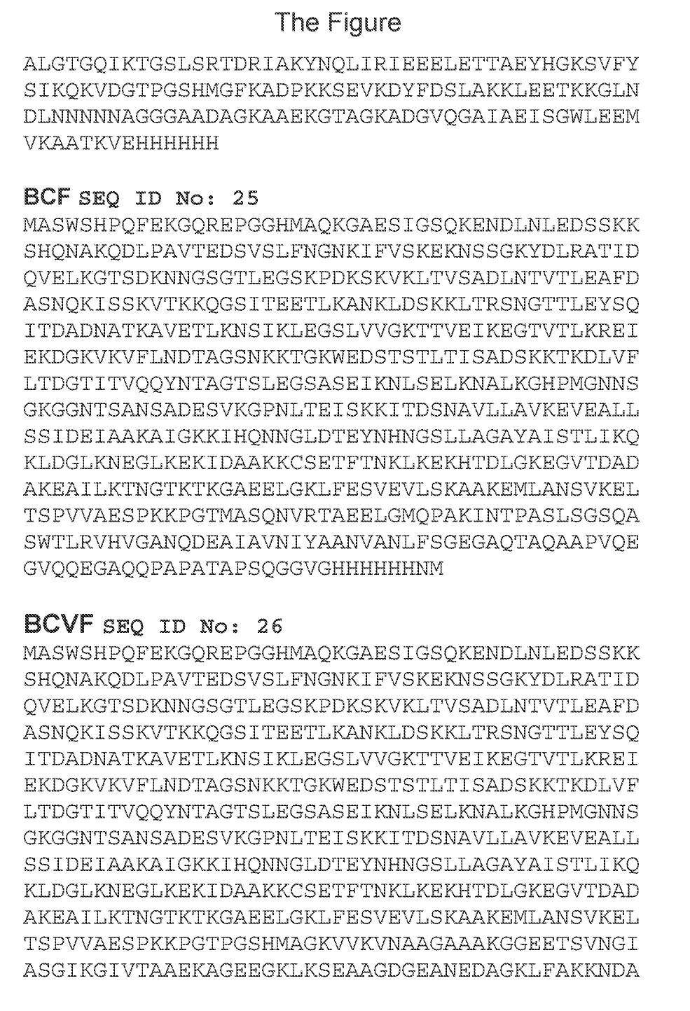

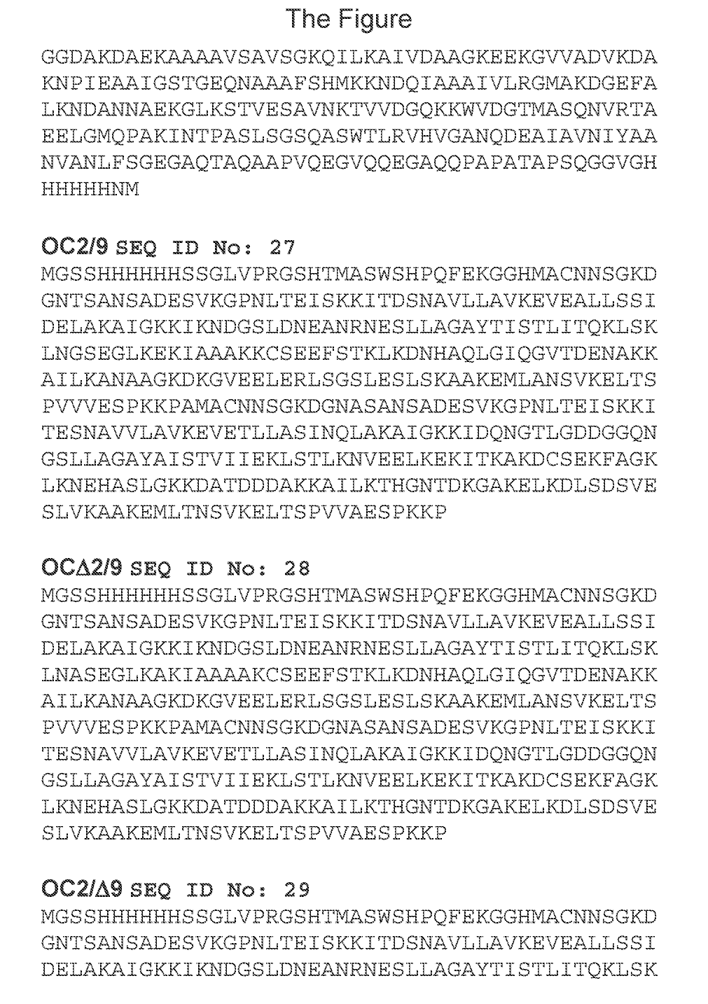

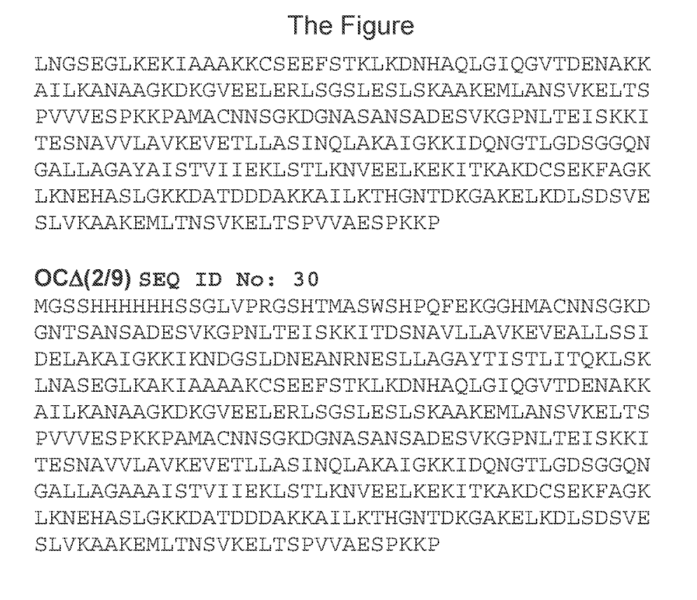

### RELATED APPLICATIONS

This application is the Continuation-In-Part of International Application No. PCT/US/2015/054303, filed Oct. 6, 2015, which claims the benefit of U.S. Provisional Application No. 62/076,059, filed Nov. 6, 2014, and U.S. Provisional Application No. 62/060,867, filed Oct. 7, 2014. This application also claims the benefit of U.S. Provisional Application No. 62/365,937, filed Jul. 22, 2016. The entire teachings of the above applications are incorporated herein by reference.

### GOVERNMENT SUPPORT

This invention was made with government support under CK000150 from Centers for Disease Control (CDC). The government has certain rights in the invention.

### INCORPORATION BY REFERENCE OF MATERIAL IN ASCII TEXT FILE

This application incorporates by reference the Sequence Listing contained in the following ASCII text file being submitted concurrently herewith:
a) File name: 43381001004_SEQUENCELISTING.txt; created Apr. 6, 2017, 120 KB in size.

### BACKGROUND

Lyme disease is the most common vector-borne disease in North America and Europe, and its range and incidence are increasing. Human Lyme disease is caused by several members of a group of closely related spirochetes belonging to the Borrelia burgdorferi sensu lato species complex. The spirochete is transmitted to humans via ticks of the genus Ixodes (Steere, A. C., N. Engl. J. Med. 1989; 321:586-96). It is a progressive multisystem disorder characterized by an initial cutaneous infection that can spread early in infection to secondary sites that include the nervous system, heart and joints (Masuzawa, T. et al., Microbiol. Immunol. 1996; 40:539-45; Stanek, G., Infection 1991; 19:263-7). The accurate diagnosis and treatment of Lyme disease depends on correlating objective clinical abnormalities with serological evidence of exposure to B. burgdorferi.

### SUMMARY OF THE INVENTION

The present invention encompasses novel recombinant Borrelia burgdorferi proteins and their use in highly sensitive and specific methods of diagnosing Lyme disease in a subject. In particular, the present invention is drawn to recombinant Borrelia fusion protein constructs comprising at least three recombinant Borrelia burgdorferi protein antigens. These constructs are also referred to herein as Borrelia chimeric protein antigens, or Borrelia chimeras. The fusion protein constructs are described in Tables 4, 5, 6 and 8 herein.

The present invention is also drawn to methods of using one, or more, or all, of these recombinant Borrelia fusion proteins in assays for assessing test samples for detecting the presence of antibodies to Borrelia burgdorferi in the test sample. For example, the Borrelia fusion proteins described herein (e.g., Tables 4, 5, 6 and 8) can be used to prepare protein arrays, or microarrays, for use in the detection methods, as well as in ELISA, other immunological assays, or any assay method capable of detecting antibody-antigen binding or antibody-antigen binding complexes. In addition to the use of the Borrelia fusion proteins of Tables 4, 5, 6 and 8 in these detection assays, one, or more, additional recombinant Borrelia antigens can be included in such assays or microarrays, such as the recombinant Borrelia antigens listed in Table 1. In one embodiment of the present invention, the microarray comprises the 25 recombinant Borrelia antigens and chimeric proteins of Table 6. In another embodiment, the microarray comprises the 11 recombinant Borrelia antigens and chimeric proteins of Table 8.

Further encompassed by the present invention are methods of diagnosing Lyme disease in a subject (such as a mammal, including a human) by assessing a test sample obtained from the subject for antibodies reactive with one, or more of the recombinant Borrelia burgdorferi fusion proteins of Tables 4, 5, 6 or 8, or, additionally one, or more, of the recombinant Borrelia antigens listed in Table 1, wherein the detection of antibody-antigen reactions (such as antibody-antigen bound complexes, also referred to herein as antibody-antigen reaction products) are indicative of Lyme disease in the individual. Such assays to detect antibody-antigen complexes are well-known to those of skill in the art. In a particular embodiment of the present invention, microarrays are used in the detection/diagnostic assays, wherein the microarrays comprise the recombinant Borrelia chimeric proteins described herein as in Tables 4, 5, 6 and 8, and also recombinant Borrelia antigens described in Table 1. In one embodiment of the present invention, the microarray comprises the 25 recombinant Borrelia antigens and chimeric proteins of Table 6. In another embodiment, the microarray comprises the 11 recombinant Borrelia antigens and chimeric proteins of Table 8.

As a result of this discovery, highly sensitive and specific methods and microarrays are now available for the assessment of a test sample for the presence of antibodies to proteins of Borrelia. The presence of such antibodies is diagnostic for Lyme disease. In addition, methods are available to identify potential diagnostic and vaccine candidates relating to Lyme disease.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

The FIGURE depicts the amino acid sequences of the chimeric antigens of Table 6 (SEQ ID NOS: 3-27) and variants of OC2/9 (SEQ ID NOS: 28-30).

### DETAILED DESCRIPTION OF THE INVENTION

A description of example embodiments of the invention follows.

Accurate, reliable diagnostic assays for Lyme disease are critical to ensure successful treatment and recovery. However, serodiagnosis of the disease is particularly difficult due to the high level of genetic heterogeneity of B. burgdorferi isolates, even among those collected from a single location. Furthermore, current serodiagnostic tests for Lyme disease lack sensitivity and specificity for detecting B. burgdorferi infection in the early stages of the disease. In the present invention, 48 highly antigenic Borrelia proteins have been identified. These recombinant Borrelia protein antigens are recognized by the majority of all sera samples analyzed including sera from patients with early Lyme disease. Moreover, this library of highly antigenic Borrelia proteins has been used to develop a set of recombinant Borrelia chimeric antigen proteins as shown in Tables 4, 5 and 6 and a highly sensitive and specific recombinant protein-based assay containing the 25 recombinant and chimeric antigens of Table 6, or the 11 recombinant antigens and recombinant chimeric antigens (Table 8) as described herein.

It has been discovered that antibodies to certain cell envelope proteins are present in sera of individuals with Lyme disease. As described herein, microarrays containing recombinant Borrelia burgdorferi antigenic proteins and chimeric antigens were prepared. The microarrays were exposed to test samples such as sera from individuals previously diagnosed with disseminated Lyme disease. Results indicated that the sera of individuals with Lyme disease reacted with specific recombinant Borrelia proteins as shown in Tables 1, 4, 5, 6 and 8. In particular, high numbers of the sera from the individuals reacted with a specific subset of those proteins as those shown in Tables 6 and 8. Results indicated that the sera of individuals with Lyme disease reacted with the recombinant and chimeric Borrelia proteins, shown in Tables 1 and 4, and 5 and in particular with the highly immunogenic proteins, shown in Tables 6 and 8. The overall sensitivity and specificity of the Lyme disease assay as described herein is shown in Tables 2, 3, 7, 9 and 10.

In certain methods and the microarrays of the invention, one or more recombinant cell envelope protein antigens, recombinant antigens and/or chimeric antigens (fusion protein constructs) are used. In certain embodiments, a set of two or more antigens are used. Other embodiments encompassed herein include three antigens, four antigens, five antigens and so forth, up to the 11 antigens of Table 8, the 16 antigens of Table 4, the 25 antigens of Table 6, the 96 antigens of Table 5 or the 48 antigens of Table 1, or all of the antigens from the afore-mentioned Tables. Representative sets include the set of proteins shown in Tables 1, 4, 5, 6, and 8. Such sets can further include homologs and paralogs of the cell envelope proteins. In one particular embodiment, the set consists essentially of the antigens set forth in Table 6. In another particular embodiment, the set consists essentially of the antigens set forth in Table 8.

In further methods and the microarrays of the invention, one or more other proteins as identified herein are used, other combinations of proteins are used (e.g., proteins selected from any of Tables 1, 4, 5, 6 and 8, in combination). Such combinations include sets of two or more, three or more, four or more, five or more, six or more, seven or more, eight or more, or other groups of proteins (e.g., with one or more selected from Table 1, Table 5 and/or Table 6).

In another embodiment of the invention, a test sample from an individual is assessed for the presence of antibodies to one or more proteins (e.g., recombinant or chimeric antigens) of B. burgdorferi). The “test sample” is a sample of blood, serum, cerebrospinal fluid, or other appropriate biological fluid from the individual. In the methods, the test sample is assessed for the presence of antibodies to one or more proteins using routine methods established in the art. In one particular embodiment, the assessment is performed using a microarray of Borrelia proteins. In certain methods, for example, an array, eg. a microarray as described below, or a protein or set of proteins as described herein, is exposed to the test sample from the individual, and any resultant binding of antibodies (if present in the test sample) to the proteins is determined. The presence of antibodies bound to (also referred to herein as “reactive with”) one, or more, antigens is indicative of antibodies specific to those proteins of B. burgdorferi. The presence of such antibodies is diagnostic for Lyme disease in the individual from whom the test sample was obtained.

The assay methods described herein encompass arrays of B. burgdorferi antigens. In one embodiment, the array comprises a subset of recombinant proteins of B. burgdorferi, such as the set the proteins set forth in Tables 1, 4, 5, 6 or 8. In other embodiments, other arrays include various subsets of proteins of B. burgdorferi, such as sets of two or more, four or more, six or more, eight or more, or other groups of antigenic proteins as set forth in Tables 1, 5 or 6. In one particular embodiment, the microarray consists essentially of all of the proteins set forth in Table 6, or consists of all of the proteins set forth in Table 6. In another particular embodiment, the microarray consists essentially of all of the proteins set forth in Table 8, or consists of all of the proteins of Table 8. In further embodiments, other microarrays include various subsets of proteins of B. burgdorferi, such as subsets of the proteins set forth in Table 1 or in Table 5 (e.g., sets of two or more, four or more, six or more, eight or more, or other groups of proteins as set forth in Table 6 or Table 8).

Other embodiments of the present invention encompass methods for the evaluation/assessment of other or additional proteins of B. burgdorferi as potential candidates for development of a diagnostic test for Lyme disease, and also for assessment of proteins of B. burgdorferi as potential candidates for development of vaccines to protect against Lyme disease. In these methods, one or more proteins of B. burgdorferi, such as described herein in Table 6 or Table 8 (e.g., in a microarray as described above), are exposed to sera from one or more individuals known to have Lyme disease, and the proteins to which antibodies from the sera bind are then determined. For example, Cy5 intensity/Cy3 intensity ratio of fluorescence, as described in the Examples, can be used. The ratio of any proteins greater than the mean ratio of the reactivity of the Lyme sera to a negative control plus three times the standard deviation indicates significant interactions between antibodies present in the Lyme sera and the B. burgdorferi protein. It is reasonable to believe that such proteins are useful for vaccine compositions to elicit an immune response in a subject, as described below. Such proteins are also proteins which can be used in diagnostic tests for Lyme disease (e.g., in the methods described above), as well as in microarrays as described herein, and additionally can be used as potential vaccine candidates.

The proteins identified herein as reacting with sera of individuals with Lyme disease are useful as vaccine immunogens against Borrelia infection. Thus, the present invention is also drawn to pharmaceutical compositions which can be used to vaccinate and/or treat Borrelia infection in an animal or human. In a particular embodiment, the pharmaceutical composition comprises one, or more, Borrelia burgdorferi antigen, such as one shown in Tables 5, 6 or 8, or another Borrelia burgdorferi antigen, such as one shown in Table 1, or a protein derived from a cell envelope protein or other protein (e.g., a protein having modifications such as insertions, deletions, or other alterations, or a protein that forms part of a chimeric protein, such as those described in U.S. Pat. Nos. 6,248,562; 7,008,625; 7,060,281; and 7,179,448, the entire teachings of which are incorporated herein by reference). Combinations of the proteins described herein (e.g., those in Tables 1, 5, 6 and 8) can also be used.

The pharmaceutical composition can also be administered together with a physiologically-acceptable carrier, an excipient and/or an adjuvant. Suitable adjuvants are well known in the art (see for example PCT Publication WO 96/40290, the entire teachings of which are incorporated herein by reference), and can be used, for example, to enhance immunogenicity, potency or half-life of the proteins in the treated animal.

The pharmaceutical compositions used to vaccinate and/or treat Borrelia infection can be prepared using methods for preparing vaccines which are well known in the art. For example, the proteins described herein can be isolated and/or purified by known techniques, such as by size exclusion chromatography, affinity chromatography, ion exchange chromatography, preparative electrophoresis, selective precipitation or combinations thereof. The prepared proteins can be mixed with suitable other reagents as described herein, such that the protein is at a suitable concentration. The dosage of the protein will vary and depends upon the age, weight and/or physical condition of the animal, e.g., mammal, human, to be treated. The optimal dosage can be determined by routine optimization techniques, using suitable animal models.

Administration of the pharmaceutical composition to be used as a vaccine can be by any suitable technique. Suitable techniques for administration of the pharmaceutical composition include, but are not limited to, injection, e.g., subcutaneous injection, intramuscular injection, intravenous injection, intra peritoneal injection; mucosal administration, e.g., exposing nasal mucosa to nose drops containing the proteins of the present invention; oral administration; and DNA immunization.

In addition, the proteins identified herein as reacting with sera of individuals with Lyme disease (e.g., those shown in Table 1 and/or Table 4 and/or Table 5, and in particular Tables 6 and 8) as well as the methods described herein can be used as prognostic markers enabling one skilled in the art to tailor treatment for disease by targeting those specific proteins. For example, should an individual's serum demonstrate reactivity with a particular subset of proteins, therapy can be initiated to reduce and/or eliminate the presence of those proteins in the individual, as shown by reducing and/or eliminating reactivity of the individual's serum with those proteins.

The present invention is also drawn to diagnostic and/or prognostic kits which comprise the proteins described herein (e.g., in a microarray as described above). The kit also includes reagents for detecting antibody-antigen complexes that are formed between the protein and antibodies that are present in a sample, e.g., a user-supplied host sample.

The specific embodiments of the present invention are described in the following examples, which are not to be interpreted as limiting in any way.

### EXAMPLE 1

### Discovery of 48 Borrelia Antigens

Protein arrays. A total of 416 genes were identified in the B. burgdorferi genome comparison. Of these, 350 (84%) produced a product that was the correct size when PCR was performed, and all 350 were successfully cloned into the T7 expression vector pET28b. Sequence confirmed plasmids were expressed using the overnight autoexpression system; expressed proteins were purified using IDA resin and printed onto nitrocellulose coated FAST slides (see for example, U.S. Ser. No. 12/784,584 and U.S. Ser. No. 12/989,003, the teaching of which are incorporated herein). In addition, representatives of several different OspC types were amplified from human isolates. The OspC types included in this study were types A, B, C, D, E, F, G, H, I, J, K and U. This study also included the highly antigenic B31 cell envelope proteins that were identified in an earlier protein array study (Xu Yet al, 2008). In total, the arrays contained approximately 400 samples including expressed Borrelia proteins, negative controls and blanks. Arrays were probed with sera from Lyme disease patients and antibodies were visualized with Cy5-conjugated goat antihuman lgG or Cy5-conjugated goat anti-human lgM to determine antibody isotype (Xu Y et al, 2008).

Serum samples. For earlier array studies, sera of patients with Lyme disease were obtained from the Centers for Disease Control (CDC). The CDC samples included sera from 31 patient collected upon initial presentation (Samples C=0 days) and at 10 days (Samples D), 20 days (Samples E), 30 days (Samples F), 60 days (Samples G), and 90 days (Samples H) post presentation. Fourteen late Lyme samples (Late) from patients who exhibited late clinical manifestations (Lyme arthritis or neuroborreliosis) obtained from the Lyme Disease Center at Stony Brook University were also analyzed. A total of 115 samples were analyzed that included 21 Sample C, 29 Sample D, 15 Sample E, 8 Sample F, 1 Sample G and12 Sample H. Normal control sera (n=14) were obtained from healthy donors.

To further assess protein arrays, sera were obtained from Dr. Mark Soloski, Johns Hopkins School of Medicine, Baltimore, Md. The samples included sera from 50 patients collected upon initial presentation (sample V1=diagnosed early Lyme, prior to treatment) and at three weeks (Samples V2=three weeks following diagnosis/and begin of treatment), seven weeks (Samples V3=seven weeks following diagnosis/treatment), and fifteen to seventeen weeks (Samples V4=˜15-17 weeks following diagnosis/treatment) post presentation. A total of 46 samples per group were analyzed. For specificity studies, sera were obtained from patients with diseases associated with serological responses that are known to produce cross-reactivity in Lyme disease tests. These included patients with syphilis, lupus, and rheumatoid arthritis. Normal control sera were obtained from healthy donors.

Results. Approximately 140 of the arrayed proteins delectably elicited an antibody response in humans with natural infections using both secondary antibodies. Of these 140 antigens, 80 proteins were recognized by at least half of the sera samples from each time point. Importantly, the majority of sera recognized at least one antigen from a subset of 48 highly antigenic Borrelia proteins using lgM and lgG secondary antibody (Table 1). This high positivity included all sera that were collected when the patient was first seen for their erythema migrans. These 48 protein's Cy5/Cy3 ratios were among the highest observed in the arrays, averaging greater than five times the standard deviation above the mean ratio of the reactivity of the Lyme sera to the negative control. In addition, a BLASTx search with highly immunogenic Borrelia proteins against the NCBI non-redundant protein database excluding all recorded Borrelia sequence revealed no significant matches to other organisms. Interestingly, 40 of the 48 antigens are plasmid encoded and the majority of encoded proteins are outer surface proteins, lipoproteins or putative outer membrane proteins. Over 90% of plasmid gene sequences have no significant homologs to non-B. burgdorferi genes. The uniqueness of plasmid sequences suggests that they serve specialized adaptive roles for Borrelia's survival and propagation in nature and in a wide variety of hosts. Furthermore, the high numbers of outer surface lipoproteins encoded by the plasmids in conjunction with the variability of plasmid sequences supports the view that these sequences are directly involved in parasite-host interactions, and therefore, are more than likely to be key immunogens (Casjens et al, 2000).

TABLE 1
48 Highly Antigenic Borrelia proteins.

| Locus | Protein name |
|---|---|
| BB_0024 | hypothetical protein |
| BB_0067a | hypothetical protein |
| BB_0312 | chemotaxis protein CheW |
| BB_0337 | enolase |
| BB_0364 | methylglyoxal synthase |
| BB_0546 | hypothetical protein |
| BB_0744 | antigen, p83/100 |
| BB_0858 | hypothetical protein |
| BB_A14 | hypothetical protein |
| BB_A16 | outer surface protein B (OspB) |
| BB_A60 | surface lipoprotein P27 |
| BB_A65 | lipoprotein |
| BB_A66 | outer surface protein |
| BB_A73 | putative antigen P35 |
| BB_A74 | outer membrane porin OMS28 |
| BB_A70 | hypothetical protein |
| BB_B07 | alpha3-beta1 integrin-binding protein |
| BB_C08 | hypothetical protein |
| BB_E09 | protein p23 |
| BB_E19 | hypothetical protein |
| BB_G01 | hypothetical protein |
| BB_H18 | hypothetical protein |
| BB_H37 | hypothetical protein |
| BB_I16 | repetitive antigen A |
| BB_I38 | hypothetical protein |
| BB_J01 | hypothetical protein |
| BB_J23 | hypothetical protein |
| BB_K07 | hypothetical protein |
| BB_N26 | hypothetical protein |
| BB_R22 | hypothetical protein |
| BB_S42 | BapA protein |
| BbuJD1_F28 | VLS-like protein |
| BbuJD1_F31 | VLS-like protein |
| BbuJD1_S13 | hypothetical protein |
| BbuN40_Q42 | Erp26 protein |
| BbuN40_V37 | Erp25 protein |
| BbuN40_X36 | Erp27 protein |
| Bbu297_B19 | outer surface protein C (OspC) |
| Bbu297_F32 | VLS-like protein |
| Bbu297_F33 | VLS-like protein |
| Bbu297_J03 | hypothetical protein |
| Bbu297_J03a | hypothetical protein |
| Bbu297_J05 | hypothetical protein |
| Bbu297_M38 | bbK2.10 protein precursor |
| Bbu297_P39 | Erp42 protein |
| Bbu297_R41 | MlpL (multicopy lipoproteins) |
| Bbu297_W44 | hypothetical protein |
| Bbu297_Y03 | hypothetical protein |

The sensitivity of the assay described herein far exceeds that of commercially available Lyme disease assays in patients in the early stages of the disease. The sensitivity of the present assay was compared with the 48 Borrelia antigens with two-tier testing using 29 of the early Lyme disease patients. Among the 29 patients with EM, the sensitivity of our protein array with lgM or lgG secondary antibody was 96% at initial presentation (Samples V1) and 97% during convalescence, 3 weeks later (Samples V2). In comparison, the percentages were 48% and 69%, respectively, for standard 2-tiered testing. The overall sensitivity and specificity of the Lyme assay as described herein for this set of sera specimens are shown below in Table 2 and Table 3.

TABLE 2
Serologic Responses in Well-Characterized
Lyme disease Patients: Sensitivity.

| | Sample | | | |
|---|---|---|---|---|
| | V1 = | Samples | Samples | Samples |
| | Early | V2 = | V3 = | v4 = |
| Immunoglobulin | Diagnosis^{a} | 3 weeks | 7 weeks | 15-17 weeks |
| IgM | 39/46 (85) | 42/46 (91) | 41/46 (89) | 38/44 (86) |
| IgG | 38/46 (83) | 4/46 (96) | 43/46 (93) | 39/44 (89) |
| IgM + IgG | 33/46/(72) | 41/46 (89) | 38/46 (83) | 34/44 (77) |
| IgM or IgG | 44/46 (96) | 45/46 (98) | 46/46 (100) | 43/44 (98) |

^{a}= number positive/total (%).

TABLE 3
Serologic Responses in Normal Controls and Potentially
Cross Reactive Patients: Specificity

| Immunoglobulin | Normal (%)^{a} | Sick-non-Lyme (%) |
|---|---|---|
| IgM | 40/40/(100) | 26/28 (89) |
| IgG | 40/40 (100) | 24/28/(86) |
| IgM + IgG | 40/40 (100) | 27/28 (96) |
| IgM or IgG | 40/40/(100) | 26/28 (93) |

^{a}= number negative/total (%).

### EXAMPLE 2

### Protein Arrays using Recombinant Chimeric Borrelia Proteins

Overview. A library of chimeric proteins using 48 highly antigenic Borrelia proteins was designed for use in developing a recombinant chimera based diagnostic assay for Lyme disease. Construction of recombinant chimeras containing genes from several genospecies allows one to generate one protein that confers antigenicity to multiple strains, revealing the great potential and adaptability of this technique. In addition, the use of pure protein preparations as antigens offers greater flexibility in adapting the test to different assay formats. It is reasonable to believe that an assay of recombinant chimeric proteins will present superior sensitivity in detecting antibodies against B. burgdorferi for the early stages of the disease, and equivalent sensitivity for the late stages of the disease, when compared to the best whole-cell assays.

Chimeric Proteins. From our library of 48 highly antigenic Borrelia proteins, we constructed 16 sets of chimeric proteins each containing three of the Borrelia antigens (Table 4). Each Borrelia protein was randomly assigned to one fusion construct and all 6 possible protein order combinations were synthesized to determine whether secondary structural characteristics may be affecting the presentation of protein epitopes. The 96 fusion constructs are shown in Table 5. Chimeras of three Borrelia antigens were chosen to keep the molecular mass of the fusion protein at, or less than (below) about 100 kDa to optimize protein yield, which can be significantly affected by protein size.

TABLE 4
Structural Characterization of Borrelia Fusion proteins

| Fusion Protein ID | Borrelia Proteins |
|---|---|
| 1TR 1-6 | BB_A74/BB_0744/Bbu297_W44 |
| 1TRI 7-12 | BB_H37/BB_E-0/BB_C08 |
| 1TRI 13-18 | BB_H18/BbuN40_X36/BbuJD1_S13 |
| 1TRI 19-24 | Bbu297_F33/OspC/BB_I16A |
| 1TRI 25-30 | Bbu297_P39/BB_E19/BB_K07 |
| 1TRI 31-36 | BB_A60/BB_0546/Bbu297_M38 |
| 1TRI 37-42 | BB_A65/Bbu297_Y03/BbuJD1_F28 |
| 1TRI 43-48 | BB_A73/BB_N26/BB_-312 |
| 2TRI 1-6 | BB_0024/BB_0067a/BB_A16 |
| 2TRI 7-12 | BB_0337/Bbu297_F32/BbuJD1_F31 |
| 2TRI 13-18 | BB_0364/BB_A66/BB_S42 |
| 2TRI 19-24 | BB_0858/BB_B-7/BB_G01 |
| 2TRI 25-30 | BB_A14/BB_I38/BB_J23 |
| 2TRI 31-36 | BB_A70/BB_J01/BB_$22 |
| 2TRI 37-42 | Bbu297_J03/Bbu297_J03a/Bbu297_J05 |
| 2TRI 43-48 | Bbu297_R41/BbuN40/Q42/BbuN40_V37 |

TABLE 5
Composition of the 16 set of chimeric Borrelia protein

| Proteins for | | |
|---|---|---|
| 1T Trimer | Gene Name | Trimer List |
| BB_0312 | purine-binding chemotaxis protein | 1T-1---- 1T-6 |
| BB_0546 | conserved hypothetical protein | (BB_0744, Bbu297_W44, BB_A74) |
| BB_0744 | Borrelia P83/P100 antigen | BB_0744/Bbu297_W44/BB_A74 |
| BB_A60 | surface lipoprotein P27 | BB_0744/BB_A74/Bbu297_W44 |
| BB_A65 | lipoprotein, putative | BB_A74/BB_0744/Bbu297_W44 |
| BB_A73 | putative antigen P35 | BB_A74/Bbu297_W44/BB_0744 |
| BB_A74 | outer membrane porin OMS28 | Bbu297_W44/BB_0744/BB_A74 |
| BB_C08 | TM2 domain family | Bbu297_W44/BB_A74/BB_0744 |
| BB_E09 | protein p23 | 1T-7---- 1T-12 |
| BB_E19 | PF-32 protein | (BB_H37, BB_E09, BB_C08) |
| BB_H18 | lipoprotein, putative | BB_H37/BB_E09/BB_C08 |
| BB_H37 | lipoprotein, putative | BB_H37/BB_C08/BB_E09 |
| BB_I16A | virulent strain-associated repetitive antigen A (VraA protein) | BB_E09/BB_H37/BB_C08 |
| BB_K07 | lipoprotein, putative | BB_E09/BB_C08/BB_H37 |
| BB_N26 | Borrelia orf-D family | BB_C08/BB_H37/BB_E09 |
| Bbu297_F33 | VMP-like sequence protein VlsE (Vls) | BB_C08/BB_E09/BB_H37 |
| Bbu297_M38 | bbK2.10 protein precursor | 1T-13---- 1T-18 |
| Bbu297_P39 | Erp42 protein | (BbuN40_X36, BB_H18, BbuJD1_S13) |
| Bbu297_W44 | conserved hypothetical protein | BbuN40_X36/BB_H18/BbuJD1_S13 |
| Bbu297_Y03 | hypothetical protein | BbuN40_X36/BbuJD1_S13/BB_H18 |
| BbuJD1_F28 | vls recombination cassette Vls10 | BB_H18/BbuN40_X36/BbuJD1_S13 |
| BbuJD1_S13 | conserved hypothetical protein | BB_H18/BbuJD1_S13/BbuN40_X36 |
| BbuN40_X36 | Erp27 protein | BbuJD1_S13/BB_H18/BbuN40_X36 |
| ospC K | | BbuJD1_S13/BbuN40_X36/BB_H18 |
| | | 1T-19---- 1T-24 |
| | | (Bbu297_F33, ospC, BB_I16A) |
| | | Bbu297_F33/ospC/BB_I16A |
| | | Bbu297_F33/BB_I16A/ospC |
| | | ospC/Bbu297_F33/BB_I16A |

ospC/BB_I16A/Bbu297_F33
BB_I16A/ospC/Bbu297_F33
BB_I16A/Bbu297_F33/ospC
1T-25---- 1T-30
(Bbu297_P39, BB_E19, BB_J47)
Bbu297_P39/BB_E19/BB_J47
Bbu297_P39/BB_J47/BB_E19
BB_E19/BB_J47/Bbu297_P39
BB_E19/Bbu297_P39/BB_J47
BB_J47/BB_E19/Bbu297_P39
BB_J47/Bbu297_P39/BB_E19
1T-31---- 1T-36
(BB_A60, BB_0546, Bbu297_M38)
BB_A60/BB_0546/Bbu297_M38
BB_A60/Bbu297_M38/BB_0546
BB_0546/BB_A60/Bbu297_M38
BB_0546/Bbu297_M38/BB_A60
Bbu297_M38/BB_0546/BB_A60
Bbu297_M38/BB_A60/BB_0546
1T-36---- 1T-42
(BB_A65, Bbu297_Y03, BbuJD1_F28)
BB_A65/Bbu297_Y03/BbuJD1_F28
BB_A65/BbuJD1_F28/Bbu297_Y03
Bbu297_Y03/BB_A65/BbuJD1_F28
Bbu297_Y03/BbuJD1_F28/BB_A65
BbuJD1_F28/Bbu297_Y03/BB_A65
BbuJD1_F28/BB_A65/Bbu297_Y03
1T-43---- 1T-48
(BB_A73, BB_N26, BB_0312)
BB_A73/BB_N26/BB_0312
BB_A73/BB_0312/BB_N26
BB_N26/BB_A73/BB_0312
BB_N26/BB_0312/BB_A73
BB_0312/BB_N26/BB_A73
BB_0312/BB_A73/BB_N26

| Proteins for | | |
|---|---|---|
| 2T Trimer | Gene Name | Trimer List |
| BB_0024 | conserved hypothetical protein | 2T-1---- 2T-6 |
| BB_0067a | peptidase, putative | (BB_0024, BB_0067a, BB_A16) |
| BB_0337 | phosphopyruvate hydratase | BB_0024/BB_0067a/BB_A16 |
| BB_0364 | methylglyoxal synthase | BB_0024/BB_A16/BB_0067a |
| BB_0858 | hypothetical protein | BB_0067a/BB_0024/BB_A16 |
| BB_A14 | Borrelia orf-D family | BB_0067a/BB_A16/BB_0024 |
| BB_A16 | outer surface protein B (OspB) | BB_A16/BB_0024/BB_0067a |
| BB_A66 | outer surface protein | BB_A16/BB_0067a/BB_0024 |
| BB_A70 | conserved hypothetical protein | 2T-7---- 2T-12 |
| BB_B07 | putative alpha3-beta1 integrin-binding protein | (BB_0337, Bbu297_F32, BbuJD1_F31) |
| BB_G01 | putative lipoprotein | BB_0337/Bbu297_F32/BbuJD1_F31 |
| BB_I38 | putative surface antigen | BB_0337/BbuJD1_F31/Bbu297_F32 |
| BB_J01 | conserved hypothetical protein | Bbu297_F32/BB_0337/BbuJD1_F31 |
| BB_J23 | tetratricopeptide repeat domain protein | Bbu297_F32/BbuJD1_F31/BB_0337 |
| BB_R22 | conserved hypothetical protein | BbuJD1_F31/BB_0337/Bbu297_F32 |
| BB_S42 | BapA protein | BbuJD1_F31/Bbu297_F32/BB_0337 |
| Bbu297_F32 | VlsF2 (Vls) | 2T-13---- 2T-18 |
| Bbu297_J03 | virulent strain associated lipoprotein | (BB_0364, BB_A66, BB_S42) |
| Bbu297_J03a | | BB_0364/BB_A66/BB_S42 |
| Bbu297_J05 | lipoprotein, putative | BB_0364/BB_S42/BB_A66 |
| Bbu297_R41 | MlpL | BB_A66/BB_0364/BB_S42 |
| BbuJD1_F31 | vmp-like sequence (Vls) protein | BB_A66/BB_S42/BB_0364 |
| BbuN40_Q42 | Erp26 protein | BB_S42/BB_0364/BB_A66 |
| BbuN40_V37 | Erp25 protein | BB_S42/BB_A66/BB_0364 |
| | | 2T-19---- 2T-24 |
| | | (BB_0858, BB_B07, BB_G01) |
| | | BB_0858/BB_B07/BB_G01 |
| | | BB_0858/BB_G01/BB_B07 |
| | | BB_B07/BB_0858/BB_G01 |
| | | BB_B07/BB_G01/BB_0858 |
| | | BB_G01/BB_0858/BB_B07 |
| | | BB_G01/BB_B07/BB_0858 |
| | | 2T-25---- 2T-30 |
| | | (BB_A14, BB_I38, BB_J23) |

BB_A14/BB_I38/BB_J23
BB_A14/BB_J23/BB_I38
BB_I38/BB_A14/BB_J23
BB_I38/BB_J23/BB_A14
BB_J23/BB_A14/BB_I38
BB_J23/BB_I38/BB_A14
2T-31---- 2T-36
(BB_A70, BB_J0, BB_R22)
BB_A70/BB_J0/BB_R22
BB_A70/BB_R22/BB_J0
BB_J01/BB_A70/BB_R22
BB_J01/BB_R22/BB_A70
BB_R22/BB_A70/BB_J01
BB_R22/BB_J01/BB_A70
2T-37---- 2T-42
(Bbu297_J03, Bbu297_J03a, Bbu297_J05)
Bbu297_J03/Bbu297_J03a/Bbu297_J05
Bbu297_J03/Bbu297_J05/Bbu297_J03a
Bbu297_J03a/Bbu297_J03/Bbu297_J05
Bbu297_J03a/Bbu297_J05/Bbu297_J03
Bbu297_J05/Bbu297_J03/Bbu297_J03a
Bbu297_J05/Bbu297_J03a/Bbu297_J03
2T-43---- 2T-48
(Bbu297_R41, BbuN40_Q42, BbuN40_V37)
Bbu297_R41/BbuN40_Q42/BbuN40_V37
Bbu297_R41/BbuN40_V37/BbuN40_Q42
BbuN40_Q42/Bbu297_R41/BbuN40_V37
BbuN40_Q42/BbuN40_V37/Bbu297_R41
BbuN40_V37/Bbu297_R41/BbuN40_Q42
BbuN40_V37/BbuN40_Q42/Bbu297_R41

Cloning of recombinant fusion proteins. Borrelia genes encoding the 48 B. burgdorferi antigens were amplified using a unique gene-specific primer pair that we specifically designed for chimeric protein construction. The primers were designed from the genomic sequence of B. burgdorferi strains and each contains a novel DNA cassettes coding for the recognition sequence for three restriction endonucleases that are in-frame with gene coding sequence. The 5′ primer (5′-AATTGGTACCCCAGGATCCCATATG+15MER ORF specific sequence) (SEQ ID NO:1) contains KpnI (underlined), BamHI (bold) and NdeI (italics) recognition sequence. The 3′ primer (5′GCGGGATCCGGTACCGTCGAC+15mer ORF specific sequence) (SEQ ID NO:2) contains BamHI, KpnI and Sall (dashed underline) recognition sequences. For amplification, ten ng of genomic DNA were used as template in a 50-μI PCR reaction containing two ORF-specific primer pairs. To increase the solubility properties of expressed cell envelope proteins, primer sets were designed to amplify coding regions without a membrane anchoring signal sequence (Dunn et al., 1990). PCR amplification was performed under stringent conditions with Platinum Taq DNA polymerase High Fidelity (Invitrogen) using conditions we have previously described (Xu et al., 2003). The PCR products were visualized by agarose gel electrophoresis. For quantification, the products were purified (PCR purification kit, Qiagen) and quantified by UV absorbance.

To create the library of recombinant fusion proteins comprised of three gene fragments, amplified products were cleaved with NdeI/BamHI (for l′l position fragment), or BamHI/KpnI (for 2nd position fragment) or KpnI/Sall (for 3rd position fragment). The 1st position fragments were directionally cloned into the unique NdeI and BamHI sites of the T7-based expression vector pET-28 (Novagen). This vector provides an N-terminal poly (His) affinity tag fused to the expressed proteins to aid in purification on nickel-Sepharose columns. Ligation reactions were transformed E. coli GCS competent cells and plasmids were purified using Eppendorf Perfectprep Plasmid 96 VAC Direct Bind Kit and verified by sequencing across the inserts. Plasmids containing fragment 1 now served as vectors for all subsequent cloning. For directional cloning of fragments two and three, vectors were digested with BamHI/XhoIl, and restriction digested amplicons of fragment two and three were ligated simultaneously into the digested vector. Following transformation, plasmids were purified and sequenced.

Protein expression and purification. Purified plasmids were transformed into E. coli BL21/DE3 competent cell for expression. Chimeric Borrelia proteins containing an N-terminal poly (His) affinity tag are expressed using the Overnight Express Autoinduction protocol. Induced cells are harvested by centrifugation and resuspended in 8M urea. Aliquots were run on SDS-PAGE. N-terminal poly His-tagged proteins were purified on nickel-Sepharose columns under denaturing conditions using Ni-NTA Spin Kit (Qiagen). The kit is designed for rapid screening and purification of His Tag fusion proteins. Protein concentration was determined by the measurement of the absorbance shift when Coomassie brilliant blue G-250 reacted with protein (Bio-Rad). Protein purity was checked by SDS-PAGE.

Serum samples. The antigenicity of the chimeric proteins was tested using CDC sera samples C (12 samples) and E (11 samples), and Baltimore sera samples V1 (28 samples) and V2 (23 samples), described above. In addition, 32 more sera samples were obtained from the CDC (CDC-R1). These included sera from 12 Lyme patients. 12 patients with diseases associated with cross-reactivity in Lyme disease tests and 8 normal controls. Moreover, 35 sera from patients with diseases associated with serological responses that are known to produce crossreactivity in Lyme disease tests were obtained from Bioreclamation LLC, Westbury, N.Y. These included patients with syphilis, lupus, and rheumatoid arthritis. Seven normal control sera were also obtained from healthy donors.

Microarray. For protein microarray, recombinant proteins and each of the individual 48 Borrelia antigens were printed onto nitrocellulose-coated FAST glass slides. In addition, recombinant proteins OspB-OspC-Flagellin (B-C-Fia), OspA-p39-p93 (A-39-93) and an OspC dimer comprised of OspC Type Band Type H (OC2/9) from an early study were also printed (Gomes Solecki et. al., 2000). Each slide in the arrays also contained 10 immobilized BSA spots for background determination. Proteome chips were probed with serum from patients with untreated early Lyme disease and sick non-Lyme patients using the Fast Pak protein array kit. Briefly, slides were first blocked overnight at 4° C. in protein array-blocking buffer before incubation in primary antibody (human sera and mouse anti His-Tag for quantitation) for 2 h. Antibodies were visualized with Cy5-conjugated goat anti-human lgG or Cy5-conjugated goat anti-human lgM (to detect bound human antibodies) and Cy3-conjugated goat anti mouse lgG (to quantify the amount of recombinant protein in each spot). After a 2 hr incubation, the slides are stringently washed and then scanned with an Axon GenePix 4200A microarray scanner. The raw data was captured and analyzed with Gene Pix Pro image analysis software. To minimize the variability among samples, the PMT gain was adjusted to equal1.0 in all the arrays with power setting at 50%. A global background subtraction method was used to subtract the background from each spot using the average mean intensity value of BSA from each slide (Xu et al., 2008).

Data analysis. For analysis of the data generated from the arrays with human serum, the spot was considered positive and included for further ratio analysis if the median fluorescence intensity of a spot is more than 1000 and the SNR (signal-noise-ratio) of a spot is more than 4. A ratio Cy5 intensity/Cy3 intensity (protein/His-tag) for each protein was then calculated. All experiments were conducted two times, and each protein's Cy5/Cy3 ratios will be averaged. The C5/C3 ratio of any chimeric protein greater than the mean ratio of the reactivity of the 17 sick non-Lyme patients to the protein plus three times the standard deviation indicated a significant interaction between antibodies in the Lyme sera and the immobilized protein.

Results. A serum panel composed of samples from patients with early Lyme disease was used to screen arrays of chimeric proteins to determine their sensitivity to detect anti-B. burgdorferi antibodies. Although a considerable amount of heterogeneity in reactivity of individual serum samples to the arrayed proteins was observed, there was remarkable consistency in the reactivity of individual samples to the 6 combinations of each recombinant protein. In fact, among the 288 arrayed proteins, the majority of them delectably elicited an antibody response in humans with natural infections using both secondary antibodies. Moreover, the chimeric constructs did not vary significantly in their immunoreactivities when compared to individual proteins. Although there were sample-specific responses, there was a subset of proteins recognized in common by a majority of the sera. Importantly, the majority of sera recognized at least one antigen from a subset of 25 highly antigenic Borrelia recombinant proteins and chimeric proteins using lgM and lgG secondary antibody (Table 6 SEQ ID NOS: 3-27 of The FIGURE). Such antigens represent the best candidates for the development of diagnostic tests, and arrays of these 25 antigens were used in all subsequent sensitivity and specificity assessments. Additionally, variants were made of chimeric antigen OC2/9 (SEQ ID NO: 27) and are shown as SEQ ID NOS: 28-30 of the FIGURE. These variants, as well as variants of the other chimeric antigens described herein, can be made and tested for efficacy using the procedures as described herein.

TABLE 6
List of 25 Highly Antigenic Borrelia proteins

| Protein ID | gene locus | gene name | Chimeric Borrelia proteins | SEQ ID NOS. |
|---|---|---|---|---|
| 1S-1 | BB_0312 | purine-binding | | 3 |
| | | chemotaxis protein | | |
| 1S-15 | BB_N26 | Borrelia orf-D family | | 4 |
| 1S-16 | Bbu297_F33 | VMP-like sequence | | 5 |
| | | protein VlsE (Vls) | | |
| 1S-4 | BB_A60 | surface lipoprotein P27 | | 6 |
| 1T-14 | | | BbuN40_X36/BbuJD1_S13/BB_H18 | 7 |
| 1T-23 | | | BB_I16A/ospC/Bbu297_F33 | 8 |
| 1T-25 | | | Bbu297_P39/BB_E19/BB_J47 | 9 |
| 1T-27 | | | BB_E19/BB_J47/Bbu297_P39 | 10 |
| 1T-28 | | | BB_E19/Bbu297_P39/BB_J47 | 11 |
| 1T-47* | | | BB_0312/BB_N26/BB_A73 | 12 |
| 1T-48* | | | BB_0312/BB_A73/BB_N26 | 13 |
| 1T-9* | | | BB_E09/BB_H37/BB_C08 | 14 |
| 2S-15* | BB_R22 | conserved hypothetical | | 15 |
| | | protein | | |
| 2S-16* | BB_S42 | BapA protein | | 16 |
| 2S-22* | BbuJD1_F31 | vmp-like sequence (Vls) | | 17 |
| | | protein | | |
| 2S-7* | BB_A16 | outer surface protein B | | 18 |
| | | (OspB) | | |
| 2T-16* | | | BB_A66/BB_S42/BB_0364 | 19 |
| 2T-33* | | | BB_J01/BB_A70/BB_R22 | 20 |
| 2T-4* | | | BB_0067a/BB_A16/BB_0024 | 21 |
| 2T-5* | | | BB_A16/BB_0024/BB_0067a | 22 |
| 2T-8* | | | BB_0337/BbuJD1_F31/Bbu297_F32 | 23 |
| 2T-9* | | | Bbu297_F32/BB_0337/BbuJD1_F31 | 24 |
| BCF* | | | BB_A19/BB_B19/BB0147 | 25 |
| BCVF* | | | BB_A19/BB_B19/Bbu297_F33/BB0147 | 26 |
| OC2/9 | | | BB_B19 B1/BB_B19 K | 27 |

The overall sensitivity and specificity of the Lyme assay for this set of sera specimens are shown below in Table 7. As can be readily seen from the table, protein arrays containing 25 recombinant Borrelia proteins out performed 2 tier testing using the Lyme disease serum panel described above. For example, the sensitivity of the protein array described herein was compared with two-tier testing using 29 of the early Lyme disease patients. Among the 29 patients with EM, the sensitivity of the instant protein array with lgM or lgG secondary antibody was 100% at initial presentation (Samples V1) and 87% during convalescence, 3 weeks later (Samples V2). In comparison, the percentages were 48% and 71%, respectively, for standard 2-tiered testing.

TABLE 7
Sensitivity and specificity of protein arrays
using IgM and IgG secondary antibodies.

| | Clinical | 2 Tier | Protein Array | | |
|---|---|---|---|---|---|
| | Diagnosis | status | IgM | IgG | IgM or IgG |
| | No. Positive/Total (%) | | | | |
| Sensitivity | V1 = Diagnosed | 14/29 (48) | 28/28 (100) | 24/27 (89) | 28/28 (100) |
| | early Lyme | | | | |
| | V2 = three week | 20/28 (71) | 19/23 (83) | 18/23 (78) | 20/23 (87) |
| | follow-up | | | | |
| | C = Diagnosed | 2/12 (17) | 11/12 (92) | 8/12 (67) | 12/12 (100) |
| | early Lyme | | | | |
| | E = 20 days | 7/12 (58) | 12/12 (100) | 8/11 (73) | 11/11 (100) |
| | post visit | | | | |
| | CDC R1 | 8/12 (67) | 9/12 (75) | 11/12 (92) | 11/12 (92) |
| | samples (mix) | | | | |
| | No. Negative/Total (%) | | | | |
| Specificity | Sick non-Lyme (47) | | 40/47 (85) | 40/47 (85) | 37/47 (79) |
| | Normal (15) | | 13/15 (87) | 12/15 (80) | 10/15 (67) |

### EXAMPLE 3

### Diagnostic Test for Lyme Disease

Overview. Using genomic and proteomic approaches, protein microarrays were fabricated based on multiple genotypes of Borrelia burgdorferi sensu stricto to identify antigens that may be useful in the development of a highly sensitive and specific single-tier assay that will have a wide range of specificity in detecting Lyme disease. Although the assay containing 25 recombinant Borrelia proteins described above is very accurate and sensitive, studies were designed to reduce the number of recombinant antigenic proteins to the least number while maintaining high sensitivity and specificity.

Serum samples. Sera from 70 Lyme disease patients were used to the sensitivity of protein arrays of the 25 recombinant proteins. This included 28 early Lyme patients from the CDC, Baltimore samples V1 and V2 described above, and 8 Lyme arthritis, 6 neurologic Lyme and 2 cardiac Lyme patients also from the CDC. In addition, for specificity studies, sera were obtained from 83 patients (sick non-Lyme) with diseases associated with serological responses that are known to produce cross-reactivity in Lyme disease test including syphilis, fibromyalgia, and rheumatoid arthritis. Normal control sera were obtained from 16 healthy donors from an area of endemicity and 36 healthy donors from non-endemic areas.

Microarray. For protein microarray, the 25 recombinant proteins of Table 6 were printed onto nitrocellulose-coated FAST glass slides. Proteome chips were probed with serum and analyzed as described above.

Results. A serum panel composed of 70 samples from patients with various stages of Lyme disease was used to screen arrays of chimeric proteins to determine their sensitivity to detect anti-B. burgdorferi antibodies. Although there were sample-specific responses, there was a subset of proteins recognized in common by a majority of the sera. Importantly, the majority of sera recognized 9 highly antigenic Borrelia chimeras using lgM secondary antibody and 7 chimeras using lgG secondary antibody for a total of 11 individual proteins (Table 8).

TABLE 8
11 Highly Antigenic Borrelia proteins.

| protein ID | Borrelia Proteins | SEQ ID NOS. |
|---|---|---|
| 1S-16 | Bbu297_F33 | 5 |
| 2S-16 | BB_S42 | 16 |
| 1T-9 | BB_E09/BB_H37/BB_C08 | 14 |
| 1T-27 | BB_E19/BB_K07/Bbu297_P39 | 10 |
| 1T-48 | BB_0312/BB_A73/BB_N26 | 13 |
| 2T-9 | BBu297_F32/BB_0337/BbuJD1_F31 | 24 |
| 2T-16 | BB_A66/BB_S42/BB_0364 | 19 |
| 2T-33 | BB_J01/BB_A70/BB_R22 | 20 |
| BCF | BB_A16/BB_B19/BB_0147 | 25 |
| BCVF | BB_A16/BB_B19/Bbu297_F33/BB_0147 | 26 |
| OC2/9 | BB_B19 Type B/Type H | 27 |

These 11 recombinant and chimeric antigens' Cy5/Cy3 ratios were among the highest observed in the arrays and each had a specificity of >99% using normal sera and sera from patients with other chronic infections or autoimmune diseases. The 11 antigens were printed on array slides and used for sensitivity and specificity analysis.

The overall sensitivity of our array of 11 proteins is shown in Table 9. For early Lyme patients, the sensitivity of the protein array with lgM or lgG secondary antibody was 71% for sera from the CDC and for the Baltimore samples 88% at initial presentation (Samples V1) and 88% during convalescence, 3 weeks later (Samples V2). In comparison, the sensitivity was 44% for standard 2-tiered testing for these same early Lyme patients. The sensitivity of the array was 100% for Lyme arthritis, neurologic Lyme and cardiac Lyme sera (Table 9).

TABLE 9
Serologic Responses in Well-Characterized
Lyme disease Patients: Sensitivity.

| | | | | Overall | |
|---|---|---|---|---|---|
| | Sample | | | | IgM or |
| | Category | Sample Group | IgM | IgG | IgG |
| Sensitivity | Lyme | Early Lyme-CDC | 57 | 46 | 71 |
| number | disease | Lyme arthritis | 63 | 100 | 100 |
| positive/ | (Total = 70) | Neurologic Lyme | 100 | 67 | 100 |
| total (%). | | Cardiac Lyme | 100 | 100 | 100 |
| | | Baltimore-V1 | 81 | 73 | 88 |
| | | Baltimore-V2 | 88 | 73 | 88 |
| | | Overall | 71 | 66 | 84 |

The specificity of the 11 antigen array assay (Table 10) was evaluated by testing sera from normal controls and from patient with diseases associated with serological responses that are known to produce cross-reactivity (Sick-non-Lyme) in currently used tests. Among the 52 healthy controls from areas in which Lyme disease is endemic, none had positive results with the assay. For the potential cross reactive patients 11 of 83 samples had positive lgM or lgG antibody responses in our assay. Thus, the overall specificity for the array assay was 100% for normal controls and 87% in Sick-non-Lyme patients.

TABLE 10
Serologic Responses in Normal Controls and Potentially
Cross Reactive Patients: Specificity.

| | | | | Overall | |
|---|---|---|---|---|---|
| | Sample | | | | IgM or |
| | Category | Sample Group | IgM | IgG | IgG |
| Specificity | Control: | EVB positive | 100 | 100 | 100 |
| number | Sick non- | Fibromyalgia | 100 | 100 | 100 |
| negative/ | Lyme | Influenza | 100 | 100 | 100 |
| total (%). | (total = 83) | Mononucleosis | 88 | 100 | 88 |
| | | Multiple sclerosis | 88 | 100 | 88 |
| | | Psoriasis | 100 | 80 | 100 |
| | | Rheumatoid arthritis | 92 | 100 | 85 |
| | | RF positive | 80 | 100 | 100 |
| | | SLE | 100 | 100 | 100 |
| | | Severe periodontitis | 88 | 100 | 88 |
| | | Syphilis | 92 | 92 | 92 |
| | | overall | 93 | 98 | 93 |
| | Control: | Healthy endemic | 94 | 100 | 94 |
| | Healthy | Healthy non-endemic | 97 | 97 | 94 |
| | (total = 52) | Overall | 96 | 98 | 94 |
| | overall | | 94 | 98 | 93 |

These data demonstrate that the microarray assays described herein offer superior sensitivity, specificity and precision in detecting antibodies against B. burgdorferi for not only early stages of the disease, but shows equivalent sensitivity for the late stages of the disease as well. As a result, a standardized sensitive and specific single-tier Lyme disease assay of recombinant chimeric Borrelia proteins that will potentially have a wide range of coverage and specificity against Lyme disease is now available. Furthermore, these assays will have significant commercial potential for the development of a next-generation rapid, single-tier point of care assay.

The teachings of all patents, published applications and references cited herein are incorporated by reference in their entirety:

Casjens, S., Palmer, N., van Vugt, R., Huang, W. G., Stevenson, B., Rosa, P., et al., A bacterial genome in flux: the twelve linear and nine circular extrachromosomal DNAs in an infectious isolate of the Lyme disease spirochete Borrelia burgdorferi. Mol. Microbiol., 2000; 35, 490-516.

Dunn, J. J., Lade, B. N., Barbour, A. G., Outer surface protein A (OspA) from the Lyme disease spirochete, Borrelia burgdorferi: high level expression and purification of a soluble recombinant form of OspA Protein Expr. Purif. 1990; 1:159-68.

Gomes-Solecki, M. J. C., Dunn, J. J., Luft, B. J., et al., 2000.Recombinant chimeric Borrelia proteins for diagnosis of Lyme disease. J. Clin. Microbial. 2000; 38:2530-2535.

Xu, Y., Bruno, J. F., Luft, B. J., Profiling the humoral immune response to Borrelia burgdorferi infection with protein microarrays. Microb. Path. 2008; 45: 403-407.

Xu, Y., Bruno, J. F., Luft, B. J., Detection of Genetic Diversity in Linear Plasm ids 28-3 and 36 in Borrelia burgdorfesensu stricto isolates by Subtractive Hybridization. Microb. Path. 2003; 35:269-78.

While this invention has been particularly shown and described with references to example embodiments thereof, it will be understood by those skilled in the art that various changes in form and details may be made therein without departing from the scope of the invention encompassed by the appended claims.

What is claimed is:

1. A recombinant Borrelia fusion protein construct comprising at least three Borrelia recombinant protein antigens wherein the recombinant antigens are selected from Table 1.

2. The protein fusion construct of claim 1 wherein the molecular mass of the fusion protein construct is about 100 kDa or less.

3. The Borrelia fusion construct of claim 1 where is the recombinant antigens were amplified by PCR with primers comprising SEQ ID NO:1 and SEQ ID NO:2.

4. A vector comprising the recombinant Borrelia fusion protein construct of claim 1.

5. A Borrelia recombinant fusion protein construct selected from the group of constructs of Table 5.

6. A microarray comprising one, or more, Borrelia chimeric protein antigens of Table 5.

7. The microarray of claim 6 further comprising one, or more, recombinant Borrelia protein antigens of Table 1.

8. The microarray of claim 7 further comprising one, or more, Borrelia recombinant protein antigens selected from the group consisting of OspB-OspC-Flagellin (B-C-Fla); OspA-p39-p93 (A-39-93) and OspC dimer consisting of OspC Type B and Type H (OC2/9).

9. A microarray comprising the recombinant Borrelia protein antigens and fusion protein antigens of Table 6.

10. A microarray comprising the recombinant Borrelia protein antigens and fusion protein antigens of Table 8.

11. A method of diagnosing Lyme disease comprising assessing a test sample obtained from a subject suspected of having Lyme disease for the presence of antibodies to Borrelia burgdorferi proteins in the sample, the method comprising contacting the sample with a microarray comprising one, or more of the Borrelia fusion protein antigens of Tables 5, 6 or 8 and detecting the presence of antibodies in the sample reactive to the Borrelia fusion protein antigens, wherein detecting the presence of antibodies to one, or more, Borrelia chimeric antigens indicates the presence of Lyme disease in the subject.

12. The method of claim 11 wherein the microarray consists of all of the recombinant antigens and fusion protein antigens of Table 6.

13. The method of claim 11 wherein the microarray consists of all of the recombinant antigens and fusion protein antigens of Table 8.

14. A method of diagnosing Lyme disease in a subject comprising assessing a test sample obtained from a subject suspected of having Lyme disease for the presence of antibodies to Borrelia burgdorferi proteins in the sample, the method comprising contacting the sample with a microarray comprising the recombinant Borrelia protein antigens of Table 6 and detecting the presence of antibodies in the sample reactive to the recombinant Borrelia antigens, wherein detecting the presence of antibodies to one, or more, recombinant Borrelia antigens indicates the presence of Lyme disease in the subject.

15. A method of diagnosing Lyme disease in a subject comprising assessing a test sample obtained from a subject suspected of having Lyme disease for the presence of antibodies to Borrelia burgdorferi proteins in the sample, the method comprising contacting the sample with a microarray comprising the recombinant Borrelia protein antigens of Table 8 and detecting the presence of antibodies in the sample reactive to the recombinant Borrelia antigens, wherein detecting the presence of antibodies to one, or more, recombinant Borrelia antigens indicates the presence of Lyme disease in the subject.

16. A method for detecting antibodies to Borrelia burgdorferi in a test sample comprising contacting the sample with a microarray of one, or more, recombinant Borrelia fusion protein antigens of Table 5, under conditions sufficient for the antibodies in the sample to react with the fusion protein antigens and detecting the antibody-antibody reaction.

17. The method of claim 16 wherein the microarray comprises the recombinant antigens and fusion protein antigens of Table 6.

18. The method of claim 16 wherein the microarray comprises the recombinant antigens and fusion protein antigens of Table 8.

19. A method for detecting antibodies to Borrelia burgdorferi in a test sample comprising contacting the sample with a microarray consisting of the recombinant Borrelia antigens and fusion protein antigens of Table 6, under conditions sufficient for the antibodies in the sample to react with the antigens and detecting the antigen-antibody reaction.

20. A method for detecting antibodies to Borrelia burgdorferi in a test sample comprising contacting the sample with a microarray consisting of the recombinant Borrelia protein antigens and fusion protein antigens of Table 8, under conditions sufficient for the antibodies in the sample to react with the antigens and detecting the antigen-antibody reaction.
